(19) United States
(12) Patent Application Publication
(10) Pub. No.: US 2012/0165346 A1
(43) Pub. Date: Jun. 28, 2012

(54) Compounds and their use as BACE inhibitors

(75) Inventors: Karin Kolmodin, Sodertalje (SE); Britt-Marie Swahn, Sodertalje (SE); Jacob Kihlström, Sodertalje (SE); Johan Lindström, Sodertalje (SE); Sofia Karlström, Sodertalje (SE); Marie Sundström, Sodertalje (SE); Stefan Von Berg, Sodertalje (SE)

(73) Assignee: ASTRAZENECA AB, Sodertalje (SE)

(21) Appl. No.: 13/331,231

(22) Filed: Dec. 20, 2011

Related U.S. Application Data
(60) Provisional application No. 61/425,853, filed on Dec. 22, 2010.

(51) Int. Cl. A61K 31/506; A61P 25/28; A61P 25/00; C07D 401/14; A61K 31/444

(52) U.S. Cl. 514/256; 546/256; 544/333; 514/333

(57) ABSTRACT

The present invention relates to novel compounds of formula (I) and their pharmaceutical compositions. In addition, the present invention relates to therapeutic methods for the treatment and/or prevention of Aβ-related pathologies such as Down's syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer's disease, memory loss, attention deficit symptoms associated with Alzheimer's disease, neurodegeneration associated with diseases such as Alzheimer's disease or dementia including dementia of mixed vascular and degenerative origin, pre-senile dementia, senile dementia and dementia associated with Parkinson's disease, progressive supranuclear palsy or cortical basal degeneration.

The present invention relates to novel compounds and their pharmaceutical compositions. In addition, the present invention relates to therapeutic methods for the treatment and/or prevention of Aβ-related pathologies such as Downs syndrome, fβ-amyloid angiopathy such as but not limited to cerebral amyloid angiopathy or hereditary cerebral hemorrhage, disorders associated with cognitive impairment such as but not limited to MCI (“mild cognitive impairment”), Alzheimer's disease, memory loss, attention deficit symptoms associated with Alzheimer's disease, neurodegeneration associated with diseases such as Alzheimer's disease or dementia including dementia of mixed vascular and degenerative origin, pre-senile dementia, senile dementia and dementia associated with Parkinson's disease, progressive supranuclear palsy or cortical basal degeneration.

### BACKGROUND

Several groups have identified and isolated aspartate proteinases that have β-secretase activity (Hussain et al., 1999; Lin et. al, 2000; Yan et. al, 1999; Sinha et. al., 1999 and Vassar et. al., 1999). β-secretase is also known in the literature as Asp2 (Yan et. al, 1999), Beta site APP Cleaving Enzyme (BACE) (Vassar et. al., 1999) or memapsin-2 (Lin et al., 2000). BACE was identified using a number of experimental approaches such as EST database analysis (Hussain et al. 1999); expression cloning (Vassar et al. 1999); identification of human homologs from public databases of predicted C. elegans proteins (Yan et al. 1999) and finally utilizing an inhibitor to purify the protein from human brain (Sinha et al. 1999). Thus, five groups employing three different experimental approaches led to the identification of the same enzyme, making a strong case that BACE is a β-secretase. Mention is also made of the patent literature: WO96/40885, U.S. Pat. No. 6,319,689, WO99/64587, WO99/31236, WO00/17369, WO00/47618, WO00/58479, WO00/69262, WO01/00663, and WO00/23576.

BACE was found to be a pepsin-like aspartic proteinase, the mature enzyme consisting of the N-terminal catalytic domain, a transmembrane domain, and a small cytoplasmic domain. BACE has an optimum activity at pH 4.0-5.0 (Vassar et al, 1999) and is inhibited weakly by standard pepsin inhibitors such as pepstatin. It has been shown that the catalytic domain minus the transmembrane and cytoplasmic domain has activity against substrate peptides (Lin et al, 2000). BACE is a membrane bound type 1 protein that is synthesized as a partially active proenzyme, and is abundantly expressed in brain tissue. It is thought to represent the major β-secretase activity, and is considered to be the rate-limiting step in the production of amyloid-(β-protein (Aβ). It is thus of special interest in the pathology of Alzheimer's disease, and in the development of drugs as a treatment for Alzheimer's disease.

Aβ or amyloid-β-protein is the major constituent of the brain plaques which are characteristic of Alzheimer's disease (De Strooper et al, 1999). Aβ is a 39-42 residue peptide formed by the specific cleavage of a class 1 transmembrane protein called APP, or amyloid precursor protein. Cleavage of APP by BACE generates the extracellular soluble APPβ fragment and the membrane bound CTFβ (C99) fragment that is subsequently cleaved by γ-secretase to generate Aβ peptide.

is Alzheimer's disease (AD) is estimated to afflict more than 20 million people worldwide and is believed to be the most common form of dementia. Alzheimer's disease is a progressive dementia in which massive deposits of aggregated protein breakdown products—amyloid plaques and neurofibrillary tangles accumulate in the brain. The amyloid plaques are thought to be responsible for the mental decline seen in Alzheimer's patients.

The likelihood of developing Alzheimer's disease increases with age, and as the aging population of the developed world increases, this disease becomes a greater and greater problem. In addition to this, there is a familial link to Alzheimer's disease and consequently any individuals possessing the double mutation of APP known as the Swedish mutation (in which the mutated APP forms a considerably improved substrate for BACE) have a much higher risk of developing AD, and also of developing the disease at an early age (see also U.S. Pat. No. 6,245,964 and U.S. Pat. No. 5,877,399 pertaining to transgenic rodents comprising APP-Swedish). Consequently, there is also a strong need for developing a compound that can be used in a prophylactic fashion for these individuals.

The gene encoding APP is found on chromosome 21, which is also the chromosome found as an extra copy in Down's syndrome. Down's syndrome patients tend to develop Alzheimer's disease at an early age, with almost all those over 40 years of age showing Alzheimer's-type pathology (Oyama et al., 1994). This is thought to be due to the extra copy of the APP gene found in these patients, which leads to over-expression of APP and therefore to increased levels of Aβ causing the high prevalence of Alzheimer's disease seen in this population. Thus, inhibitors of BACE could be useful in reducing Alzheimer's-type pathology in Down's syndrome patients.

Drugs that reduce or block BACE activity should therefore reduce Aβ levels and levels of fragments of Aβ in the brain, or elsewhere where Aβ or fragments thereof deposit, and thus slow the formation of amyloid plaques and the progression of AD or other maladies involving deposition of Aβ or fragments thereof (Yankner, 1996; De Strooper and Konig, 1999). BACE is therefore an important candidate for the development of drugs as a treatment and/or prophylaxis of Aβ-related pathologies such as Down's syndrome, β-amyloid angiopathy such as but not limited to cerebral amyloid angiopathy or hereditary cerebral hemorrhage, disorders associated with cognitive impairment such as but not limited to MCI (“mild cognitive impairment”), Alzheimer's disease, memory loss, attention deficit symptoms associated with Alzheimer's disease, neurodegeneration associated with diseases such as Alzheimer's disease or dementia including dementia of mixed vascular and degenerative origin, pre-senile dementia, senile dementia and dementia associated with Parkinson's disease, progressive supranuclear palsy or cortical basal degeneration.

It would therefore be useful to inhibit the deposition of Aβ and portions thereof by inhibiting BACE through inhibitors such as the compounds provided herein.

The therapeutic potential of inhibiting the deposition of Aβ has motivated many groups to isolate and characterize secretase enzymes and to identify their potential inhibitors (see, e.g., WO01/23533, EP0855444, WO00/17369, WO00/58479, WO00/47618, WO00/77030, WO01/00665, WO01/00663, WO01/29563, WO02/25276, U.S. Pat. No. 5,942,400, U.S. Pat. No. 6,245,884, U.S. Pat. No. 6,221,667, U.S. Pat. No. 6,211,235, WO02/02505, WO02/02506, WO02/02512, WO02/02518, WO02/02520, WO02/14264, WO05/058311, WO05/097767, WO06/041404, WO06/041405, WO06/0065204, WO06/0065277, US2006287294, WO06/138265, US20050282826, US20050282825, US20060281729, WO06/138217, WO06/138230, WO06/138264, WO06/138265, WO06/138266, WO06/099379, WO06/076284, US20070004786, US20070004730, WO07/011,833, WO07/011,810, US20070099875, US20070099898, WO07/058,601, WO07/058,581, WO07/058,580, WO07/058,583, WO07/058,582, WO07/058,602, WO07/073,284, WO07/049,532, WO07/038,271, WO07/016,012, WO07/005,366, WO07/005,404, WO07/149,033 and WO06/0009653.

It is desirable for BACE inhibitors to have a high degree of potency, which can be measured as the inhibition of BACE in in vitro systems.

### DISCLOSURE OF THE INVENTION

The present invention relates to a compound according to formula (I):

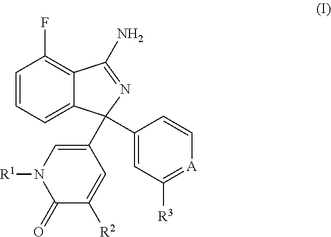

is wherein

### A is N or CR⁴;

R¹ is C₁₋₆alkyl, C₃₋₆cycloalkyl or C₁₋₆haloalkyl;
R² is C₁₋₆alkyl, C₃₋₆cycloalkyl or C₁₋₆haloalkyl;
R³ is heteroaryl, wherein said heteroaryl is optionally substituted with one or more R⁵;
R⁴ is hydrogen or halogen;
R⁵ is independently halogen, cyano, C₁₋₆alkyl, C₁₋₆haloalkyl, C₃₋₆cycloalkyl, C₂₋₆alkenyl, C₂₋₆alkynyl, OC₁₋₆alkyl or OC₁₋₆haloalkyl, wherein said C₃₋₆cycloalkyl, C₁₋₆alkyl, C₂₋₆alkenyl, C₂₋₆alkynyl is optionally substituted with one to three R⁶;
R⁶ is independently halogen or OC₁₋₆alkyl;
as a free base or a pharmaceutically acceptable salt thereof.

In one embodiment of the present invention, R¹ is C₁₋₆alkyl. In another embodiment of the invention, R¹ is C₁₋₃alkyl.

In one embodiment of the present invention, R² is C₁₋₃alkyl or C₁₋₃haloalkyl. In another embodiment of the invention, R² is C₁₋₂alkyl or trifluoromethyl.

In one embodiment of the present invention, R³ is pyridine or pyrimidine, optionally substituted with one or two R⁵.

In one embodiment of the present invention, R⁴ is hydrogen or fluoro.

In one embodiment of the present invention, R⁵ is independently halogen, cyano, C₁₋₃alkyl, C₁₋₃haloalkyl, C₂₋₆alkenyl, C₂₋₆alkynyl or OC₁₋₃alkyl, wherein said C₁₋₃alkyl, C₂₋₆alkenyl, C₂₋₆alkynyl is optionally substituted with one R⁶. In another embodiment of the invention, R⁵ is independently halogen, cyano, C₁₋₃ alkyl, C₂₋₆alkynyl or OC₁₋₃alkyl.

In one embodiment of the present invention,

### A is N or CR⁴;

R¹ is C₁₋₆alkyl or C₁₋₆haloalkyl;
R² is C₁₋₃ alkyl or C₁₋₃ haloalkyl;
R³ is heteroaryl, wherein said heteroaryl is optionally substituted with one or two R⁵;
R⁴ is hydrogen or fluoro;
R⁵ is independently halogen, cyano, C₁₋₃alkyl, C₁₋₃haloalkyl, C₂₋₆alkenyl, C₂₋₆alkynyl or OC₁₋₃alkyl, wherein said C₁₋₃alkyl, C₂₋₆alkenyl, C₂₋₆alkynyl is optionally substituted with one to three R⁶;
R⁶ is independently halogen or OC₁₋₆alkyl.

In one embodiment of the present invention,

### A is N or CR⁴;

R¹ is C₁₋₃alkyl;
R² is C₁₋₃ alkyl or C₁₋₃ haloalkyl;
R³ is heteroaryl, wherein said heteroaryl is optionally substituted with one or two R⁵;
R⁴ is hydrogen or fluoro;
R⁵ is independently halogen, cyano, C₁₋₃alkyl, C₂₋₆alkynyl or OC₁₋₃alkyl, wherein said C₁₋₃alkyl, C₂₋₆alkenyl, C₂₋₆alkynyl is optionally substituted with one to three R⁶;
R⁶ is independently halogen or OC₁₋₆alkyl.

In one embodiment of the present invention,

### A is N or CR⁴;

R¹ is C₁₋₃alkyl;
R² is C₁₋₂alkyl or trifluoromethyl;
R³ is pyridine or pyrimidine, optionally substituted with one or two R⁵;
R⁴ is hydrogen or fluoro;
R⁵ is independently fluoro, chloro, cyano, C₁₋₃alkyl, C₁₋₃haloalkyl, C₂₋₄alkenyl, C₂₋₄alkynyl or
OC₁₋₃alkyl, wherein said C₁₋₃alkyl, C₂₋₄alkenyl, C₂₋₄alkynyl is optionally substituted with one R⁶;

R⁶ is independently halogen or OC₁₋₆alkyl.

In one embodiment of the present invention, the compound of formula (I) is the S-enantiomer. In another embodiment of the present invention, the compound of formula (I) is the R-enantiomer. is In one embodiment, the compound of formula (I) has the following stereochemistry:

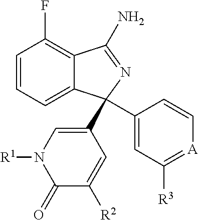

In another embodiment, the invention relates to a compound of formula (I) selected from the group consisting of:
5-(3-Amino-4-fluoro-1-(3-(5-(prop-1-ynyl)pyridin-3-yl)phenyl)-1H-isoindol-1-yl)-1,3-dimethylpyridin-2(1H)-one;
5-(3-Amino-4-fluoro-1-(5′-(prop-1-ynyl)-2,3′-bipyridin-4-yl)-1H-isoindol-1-yl)-1-ethyl-3-methylpyridin-2(1H)-one;
5-(3-amino-4-fluoro-1-(3-(5-(prop-1-ynyl)pyridin-3-yl)phenyl)-1H-isoindol-1-yl)-1-ethyl-3-methylpyridin-2(1H)-one;
5-(3-Amino-4-fluoro-1-(3-(4-fluoro-5-methylpyridin-3-yl)phenyl)-1H-isoindol-1-yl)-1-ethyl-3-methylpyridin-2(1H)-one;
5-(3-(3-Amino-4-fluoro-1-(1-isopropyl-5-methyl-6-oxo-1,6-dihydropyridin-3-yl)-1H-isoindol-1-yl)phenyl)nicotinonitrile;
5-(3-Amino-4-fluoro-1-(3-(pyrimidin-5-yl)phenyl)-1H-isoindol-1-yl)-1-ethyl-3-methylpyridin-2(1H)-one;
5-(3-Amino-4-fluoro-1-(3-(5-fluoropyridin-3-yl)phenyl)-1H-isoindol-1-yl)-1-isopropyl-3-methylpyridin-2(1H)-one;
5-(3-(3-Amino-1-(1-ethyl-5-methyl-6-oxo-1,6-dihydropyridin-3-yl)-4-fluoro-1H-isoindol-1-yl)phenyl)nicotinonitrile;
5-(3-Amino-4-fluoro-1-(3-(5-fluoropyridin-3-yl)phenyl)-1H-isoindol-1-yl)-1-ethyl-3-methylpyridin-2(1H)-one;
5-(3-(3-Amino-4-fluoro-1-(1-methyl-6-oxo-5-(trifluoromethyl)-1,6-dihydropyridin-3-yl)-1H-isoindol-1-yl)phenyl)nicotinonitrile;
5-(3-Amino-4-fluoro-1-(3-(5-methoxypyridin-3-yl)phenyl)-1H-isoindol-1-yl)-1-methyl-3-(trifluoromethyl)pyridin-2(1H)-one;
5-(3-Amino-4-fluoro-1-(3-(5-fluoropyridin-3-yl)phenyl)-1H-isoindol-1-yl)-1-methyl-3-(trifluoromethyl)pyridin-2(1H)-one;
5-(3-Amino-4-fluoro-1-(3-(pyrimidin-5-yl)phenyl)-1H-isoindol-1-yl)-1-methyl-3-(trifluoromethyl)pyridin-2(1H)-one;
5-(3-Amino-4-fluoro-1-(3-(pyrimidin-5-yl)phenyl)-1H-isoindol-1-yl)-1,3-dimethylpyridin-2(1H)-one;
5-(3-Amino-1-(5′-chloro-2,3′-bipyridin-4-yl)-4-fluoro-1H-isoindol-1-yl)-1,3-dimethylpyridin-2(1H)-one;
5-(3-Amino-4-fluoro-1-(5′-(prop-1-ynyl)-2,3′-bipyridin-4-yl)-1H-isoindol-1-yl)-1,3-diethylpyridin-2(1H)-one;
5-(3-(3-amino-1-(1,5-dimethyl-6-oxo-1,6-dihydropyridin-3-yl)-4-fluoro-1H-isoindol-1-yl)phenyl)nicotinonitrile;
5-(3-Amino-1-(3-(5-chloropyridin-3-yl)phenyl)-4-fluoro-1H-isoindol-1-yl)-1,3-dimethylpyridin-2(1H)-one;
5-(3-Amino-4-fluoro-1-(3-(5-fluoropyridin-3-yl)phenyl)-1H-isoindol-1-yl)-1,3-dimethylpyridin-2(1H)-one;
5-(3-amino-4-fluoro-1-(3-(5-methoxypyridin-3-yl)phenyl)-1H-isoindol-1-yl)-1,3-dimethylpyridin-2(1H)-one;
5-(3-Amino-4-fluoro-1-(4-fluoro-3-(5-(prop-1-ynyl)pyridin-3-yl)phenyl)-1H-isoindol-1-yl)-1,3-dimethylpyridin-2(1H)-one;
5-(3-Amino-4-fluoro-1-(3-(5-fluoropyridin-3-yl)phenyl)-1H-isoindol-1-yl)-3-(difluoromethyl)-1-ethylpyridin-2(1H)-one;
5-(3-Amino-4-fluoro-1-(3-(5-(prop-1-ynyl)pyridin-3-yl)phenyl)-1H-isoindol-1-yl)-3-(difluoromethyl)-1-ethylpyridin-2(1H)-one;
5-(3-Amino-4-fluoro-1-(3-(6-(prop-1-ynyl)pyridin-2-yl)phenyl)-1H-isoindol-1-yl)-1-ethyl-3-methylpyridin-2(1H)-one; and
5-(3-Amino-4-fluoro-1-(3-(4-(prop-1-ynyl)pyridin-2-yl)phenyl)-1H-isoindol-1-yl)-1-ethyl-3-methylpyridin-2(1H)-one;
   or a pharmaceutically acceptable salt of any foregoing compound.

In another aspect of the invention, there is provided a pharmaceutical composition comprising as active ingredient a therapeutically effective amount of a compound according formula (I), or a pharmaceutically acceptable salt thereof, in association with at least one pharmaceutically is acceptable excipient, carrier or diluent.

In another aspect of the invention, there is provided a compound according to formula (I), or a pharmaceutically acceptable salt thereof, for use as a medicament.

In another aspect of the invention, there is provided a compound according to formula (I), or a pharmaceutically acceptable salt thereof, for treating or preventing an Aβ-related pathology.

In another aspect of the invention, there is provided a compound according to formula (I), or a pharmaceutically acceptable salt thereof, for treating or preventing an Aβ-related pathology, wherein said Aβ-related pathology is Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is a compound according to formula (I), or a pharmaceutically acceptable salt thereof, for treating or preventing Alzheimer's disease.

In another aspect of the invention, there is provided use of a compound according to formula (I), or a pharmaceutically acceptable salt thereof, as a medicament for treating or preventing an Aβ-related pathology.

In another aspect of the invention, there is provided use of a compound according to formula (I), or a pharmaceutically acceptable salt thereof, as a medicament for treating or preventing an Aβ-related pathology, wherein said Aβ-related pathology is Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided use of a compound according to formula (I), or a pharmaceutically acceptable salt thereof, as a medicament for treating or preventing Alzheimer's disease.

In another aspect of the invention, there is provided use of a compound according to formula (I), or a pharmaceutically acceptable salt thereof, in the manufacture of a medicament for treating or preventing an Aβ-related pathology.

In another aspect of the invention, there is provided use of a compound according to formula (I), or a pharmaceutically acceptable salt thereof, in the manufacture of a medicament for treating or preventing an Aβ-related pathology, wherein said Aβ-related pathology is Down's syndrome, a β-amyloid angiopathy, cerebral amyloid angiopathy, hereditary cerebral hemorrhage, a disorder associated with cognitive impairment, MC1 (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided use of a compound according to formula (I), or a pharmaceutically acceptable salt thereof, in the manufacture of a medicament for treating or preventing Alzheimer's disease.

In another aspect of the invention, there is provided a method of inhibiting activity of BACE comprising contacting said BACE with a compound according to formula (I), or a pharmaceutically acceptable salt thereof.

In another aspect of the invention, there is provided a method of treating or preventing an Aβ-related pathology in a mammal, such as a human being, comprising administering to said patient a therapeutically effective amount of a compound according to formula (I), or a pharmaceutically is acceptable salt thereof.

In another aspect of the invention, there is provided a method of treating or preventing an Aβ-related pathology in a mammal, such as a human being, comprising administering to said patient a therapeutically effective amount of a compound according to formula (I), or a pharmaceutically acceptable salt thereof, wherein said Aβ-related pathology is Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of treating or preventing Alzheimer's disease, comprising administering to said patient a therapeutically effective amount of a compound according to formula (I), or a pharmaceutically acceptable salt thereof.

In another aspect of the invention, there is provided a method of treating or preventing Alzheimer's disease, comprising administering to said patient a therapeutically effective amount of a compound according to formula (I), or a pharmaceutically acceptable salt thereof, wherein said mammal is a human.

In another aspect of the invention, there is provided a method of treating or preventing an Aβ-related pathology in a mammal, such as human being, comprising administering to the patient a therapeutically effective amount of a compound according to formula (I), or a pharmaceutically acceptable salt thereof, and at least one cognitive enhancing agent, memory enhancing agent, or choline esterase inhibitor.

In another aspect of the invention, there is provided a method of treating or preventing an Aβ-related pathology in a mammal, comprising administering to said patient a therapeutically effective amount of a compound according to formula (I), or a pharmaceutically acceptable salt thereof, and at least one cognitive enhancing agent, memory enhancing agent, or choline esterase inhibitor, wherein said Aβ-related pathology is Down's syndrome, a β-amyloid angiopathy, is cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

In another aspect of the invention, there is provided a method of treating or preventing an Aβ-related pathology in a mammal, comprising administering to said patient a therapeutically effective amount of a compound according to formula (I), or a pharmaceutically acceptable salt thereof, and at least one cognitive enhancing agent, memory enhancing agent, or choline esterase inhibitor, wherein said Aβ-related pathology is Alzheimer's disease.

In another aspect, the invention relates to a pharmaceutical composition comprising (i) a compound of formula (I), or a pharmaceutically acceptable salt thereof, (ii) an additional therapeutic agent, or a pharmaceutically acceptable salt thereof, and (iii) pharmaceutically acceptable excipients, carriers or diluents.

In another aspect, the invention relates to a pharmaceutical composition comprising (i) a compound of formula (I), or a pharmaceutically acceptable salt thereof, (ii) at least one agent selected from the group consisting of cognitive enhancing agents, memory enhancing agents and choline esterase inhibitors, and (iii) pharmaceutically acceptable excipients, carriers or diluents.

The treatment of Aβ-related pathology defined herein may be applied as a mono therapy or may involve, in addition to the compound of the invention, conjoint treatment with conventional therapy of value in treating one or more disease conditions referred to herein. Such conventional therapy may include one or more of the following categories of agents: acetyl cholinesterase inhibitors, anti-inflammatory agents, cognitive and/or memory enhancing agents or atypical antipsychotic agents. Cognitive enhancing agents, memory enhancing agents and acetyl choline esterase inhibitors includes, but not limited to, donepezil (ARICEPT), galantamine (REMINYL or RAZADYNE), rivastigmine (EXELON), tacrine (COGNEX) and memantine (NAMENDA, AXURA or EBIXA). Atypical antipsychotic agents includes, but not limited to, olanzapine (marketed as is ZYPREXA), aripiprazole (marketed as ABILIFY), risperidone (marketed as RISPERDAL), quetiapine (marketed as SEROQUEL), clozapine (marketed as CLOZARIL), ziprasidone (marketed as GEODON) and olanzapine/fluoxetine (marketed as SYMBYAX).

Such conjoint treatment may be achieved by way of the simultaneous, sequential or separate dosing of the individual components of the treatment. Such combination products employ the compounds of the invention.

Additional conventional therapy may include one or more of the following categories of agents: (i) antidepressants such as agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine and equivalents and pharmaceutically active isomer(s) and metabolite(s) thereof.

(ii) atypical antipsychotics including for example quetiapine and pharmaceutically active isomer(s) and metabolite(s) thereof.

(iii) antipsychotics including for example amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof

(iv) anxiolytics including for example alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically is active isomer(s) and metabolite(s) thereof.

(v) anticonvulsants including for example carbamazepine, clonazepam, ethosuximide, felbamate, fosphenyloin, gabapentin, lacosamide, lamotrogine, levetiracetam, oxcarbazepine, phenobarbital, phenyloin, pregabaline, rufinamide, topiramate, valproate, vigabatrine, zonisamide and equivalents and pharmaceutically active isomer(s) and metabolite(s) thereof

(vi) Alzheimer's therapies including for example donepezil, rivastigmine, galantamine, memantine, tacrine and equivalents and pharmaceutically active isomer(s) and metabolite(s) thereof

(vii) Parkinson's therapies including for example deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof.

(viii) migraine therapies including for example almotriptan, amantadine, bromocriptine, butalbital, cabergoline, dichloralphenazone, dihydroergotamine, eletriptan, frovatriptan, lisuride, naratriptan, pergolide, pizotiphen, pramipexole, rizatriptan, ropinirole, sumatriptan, zolmitriptan, zomitriptan, and equivalents and pharmaceutically active isomer(s) and metabolite(s) thereof.

(ix) stroke therapies including for example thrombolytic therapy with eg activase and desmoteplase, abciximab, citicoline, clopidogrel, eptifibatide, minocycline, and equivalents and pharmaceutically active isomer(s) and metabolite(s) thereof.

(x) urinary incontinence therapies including for example darafenacin, falvoxate, oxybutynin, propiverine, robalzotan, solifenacin, tolterodine and equivalents and pharmaceutically active isomer(s) and metabolite(s) thereof.

(xi) neuropathic pain therapies including for example lidocain, capsaicin, and anticonvulsants such as gabapentin, pregabalin, and antidepressants such as duloxetine, venlafaxine, amitriptyline, klomipramine, and equivalents and pharmaceutically active isomer(s) and is metabolite(s) thereof

(xii) nociceptive pain therapies such as paracetamol, NSAIDS and coxibs, such as celecoxib, etoricoxib, lumiracoxib, valdecoxib, parecoxib, diclofenac, loxoprofen, naproxen, ketoprofen, ibuprofen, nabumeton, meloxicam, piroxicam and opioids such as morphine, oxycodone, buprenorfin, tramadol, and equivalents and pharmaceutically active isomer(s) and metabolite(s) thereof

(xiii) insomnia therapies including for example agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem and equivalents and pharmaceutically active isomer(s) and metabolite(s) thereof.

(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof.

Such combination products employ the compounds of this invention within the dosage range described herein and the other pharmaceutically active compound or compounds within approved dosage ranges and/or the dosage described in the publication reference.

The present invention relates to the use of compounds of formula (I) as hereinbefore defined as well as to the salts thereof. Salts for use in pharmaceutical compositions will be pharmaceutically acceptable salts, but other salts maybe useful in the production of the compounds of formula (I).

The definitions set forth in this application are intended to clarify terms used throughout this iii application. The term “herein” means the entire application.

All compounds in the present invention may exist in particular geometric or stereo isomeric forms. The present invention takes into account all such compounds, including cis- and trans isomers, R- and S-enantiomers, diastereomers, the racemic mixtures thereof, and other mixtures is thereof, as being covered within the scope of this invention. Additional asymmetric carbon atoms may be present in a substituent such as an alkyl group. All such isomers, as well as mixtures thereof, are intended to be included in this invention. The compounds herein described may have asymmetric centers. Compounds of the present invention containing an asymmetrically substituted atom may be isolated in optically active or racemic forms. It is well known in the art how to prepare optically active forms, such as by resolution of racemic forms, by synthesis from optically active starting materials, or synthesis using optically active reagents. When required, separation of the racemic material can be achieved by methods known in the art. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents, positions of substituents and/or variables are permissible only if such combinations result in stable compounds.

As used in this application, the term “optionally substituted,” means that substitution is optional and therefore it is possible for the designated atom or moiety to be unsubstituted.

As used herein, “alkyl”, used alone or as a suffix or prefix, is intended to include both branched and straight chain saturated aliphatic hydrocarbon groups having from 1 to 12 carbon atoms or if a specified number of carbon atoms is provided then that specific number would be intended. For to example “C₁₋₆ alkyl” denotes alkyl having 1, 2, 3, 4, 5 or 6 carbon atoms. Examples of alkyl include, but are not limited to, methyl, ethyl, n-propyl, i-propyl, n-butyl, i-butyl, sec-butyl, t-butyl, pentyl, and hexyl. In the case where a subscript is the integer 0 (zero) the group to which the subscript refers to indicates that the group may be absent, i.e. there is a direct bond between the groups.

As used herein, “alkenyl” used alone or as a suffix or prefix is intended to include both branched and straight-chain alkene or olefin containing aliphatic hydrocarbon groups having from 2 to 12 carbon atoms or if a specified number of carbon atoms is provided then that specific number would be intended. For example “C₂₋₆alkenyl” denotes alkenyl having 2, 3, 4, 5 or 6 carbon atoms. Examples of alkenyl include, but are not limited to, vinyl, allyl, 1-propenyl, 1-butenyl, 2-butenyl, 3-butenyl, 2-methylbut-2-enyl, 3-methylbut-1-enyl, 1-pentenyl, 3-pentenyl and 4-hexenyl.

As used herein, “alkynyl” used also or as a suffix or prefix is intended to include both branched and straight-chain alkynyl or olefin containing aliphatic hydrocarbon groups having from 2 to 12 carbon atoms or if a specified number of carbon atoms is provided then that specific number would be intended. Examples include, but are not limited to, ethynyl, 1-propynyl, 2-propynyl, 3-butynyl, pentynyl, hexynyl and 1-methylpent-2-ynyl.

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. Examples of polycyclic rings include, but are not limited to, 2,3-dihydro-1,4-benzodioxine and 2,3-dihydro-1-benzofuran.

As used herein, the term “cycloalkyl” is intended to include saturated ring groups, having the specified number of carbon atoms. These may include fused or bridged polycyclic systems. Cycloalkyls have from 3 to 10 carbon atoms in their ring structure, and, in one embodiment, have 3, 4, 5, and 6 carbons in the ring structure. For example, “C₃₋₆cycloalkyl” denotes such groups as cyclopropyl, cyclobutyl, cyclopentyl, or cyclohexyl.

As used herein, the term “cycloalkenyl” is intended to include unsaturated ring groups, having is the specified number of carbon atoms. These may include fused or bridged polycyclic systems. Cycloalkenyls have from 3 to 10 carbon atoms in their ring structure, and, in one embodiment, have 3, 4, 5, and 6 carbons in the ring structure. For example, “C₃₋₆cycloalkenyl” denotes such groups as cyclopropenyl, cyclobutenyl, cyclopentenyl, or cyclohexenyl.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂-group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring —NH is optionally substituted with acetyl, formyl, methyl or mesyl; and a ring is optionally substituted with one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl, tetrahydro-thiopyranyl, tetrahydro-thiopyran 1-oxide, tetrahydro-thiopyran 1,1-dioxide, 1H-pyridin-2-one, and 2,5-dioxoimidazolidinyl.

As used herein, “halo” or “halogen” refers to fluoro, chloro, bromo, and iodo.

“Counterion” is used to represent a small, negatively or positively charged species such as chloride, bromide, hydroxide, acetate, sulfate, tosylate, benezensulfonate, ammonium, lithium ion and sodium ion and the like.

As used herein, “heteroaryl” refers to a heteroaromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl is groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, benzoxazolyl, aza-benzoxazolyl indolinyl, imidazothiazolyl and the like. In some embodiments, the heteroaryl group has from 1 to 20 carbon atoms, and in further embodiments from 3 to 20 carbon atoms. In some embodiments, the heteroaryl group contains 3 to 14, 4 to 14, 3 to 7, or 5 to 6 ring-forming atoms. In some embodiments, the heteroaryl group has 1 to 4, 1 to 3, or 1 to 2 heteroatoms. In some embodiments, the heteroaryl group has 1 heteroatom.

As used herein, “haloalkyl”, used alone or as a suffix or prefix, is intended to include both branched and straight chain saturated aliphatic hydrocarbon groups, having at least one halogen substituent and having from 1 to 12 carbon atoms or if a specified number of carbon atoms is provided then that specific number would be intended. For example “C₀₋₆haloalkyl” denotes alkyl having 0, 1, 2, 3, 4, 5 or 6 carbon atoms. Examples of haloalkyl include, but are not limited to, fluoromethyl, difluoromethyl, trifluoromethyl, chlorofluoromethyl, 1-fluoroethyl, 3-fluoropropyl, 2-chloropropyl, 3,4-difluorobutyl.

As used herein, the phrase “protecting group” means temporary substituents which protect a potentially reactive functional group from undesired chemical transformations. Examples of such protecting groups include esters of carboxylic acids, silyl ethers of alcohols, and acetals and ketals of aldehydes and ketones respectively. The field of protecting group chemistry has been reviewed (Greene, T. W.; Wuts, P. G. M. Protective Groups in Organic Synthesis, 3^{rd} ed.; Wiley: New York, 1999).

As used herein, “pharmaceutically acceptable” is employed herein to refer to those compounds, materials, compositions, and/or dosage forms which are, within the scope of sound medical ici judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed is compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the non-toxic salts or the quaternary ammonium salts of the parent compound formed, for example, from non-toxic inorganic or organic acids. For example, such non-toxic salts include those derived from inorganic acids such as hydrochloric acid.

The pharmaceutically acceptable salts of the present invention can be synthesized from the parent compound that contains a basic or acidic moiety by chemical methods. Generally, such salts can be prepared by reacting the free acid or base forms of these compounds with a stoichiometric amount of the appropriate base or acid in water or in an organic solvent, or in a mixture of the two; generally, nonaqueous media like diethyl ether, ethyl acetate, ethanol, isopropanol, or acetonitrile are used.

As used herein, “tautomer” means other structural isomers that exist in equilibrium resulting from the migration of a hydrogen atom. For example, keto-enol tautomerism where the resulting compound has the properties of both a ketone and an unsaturated alcohol. Other examples of tautomerism include 7-fluoro-3H-isoindol-1-amine and its tautomer 7-fluoroisoindolin-1-imine.

It is understood that in compound representations throughout this description, only one of the possible tautomers is drawn or named.

As used herein “stable compound” and “stable structure” are meant to indicate a compound that is sufficiently robust to survive isolation to a useful degree of purity from a reaction mixture, and formulation into an efficacious therapeutic agent.

Compounds of the invention further include hydrates and solvates.

The present invention further includes isotopically-labelled compounds of the invention. An “isotopically” or “radio-labelled” compound is a compound of the invention where one or more atoms are replaced or substituted with an atom having an atomic mass or mass number different from the atomic mass or mass number typically found in nature (i.e., naturally occurring). Suitable isotopes that may be incorporated in compounds of the present invention include but are not limited to ²H (also written as D for deuterium), ³H (also written as T for tritium), ¹¹C, ¹³C ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I, and ¹³¹I. The radionuclide that is incorporated in the instant radio-labelled compounds will depend on the specific application of that radio-labelled compound. For example, for in vitro receptor labelling and competition assays, compounds that incorporate ³H, ¹⁴C, ⁸²Br, ¹²⁵I, ¹³¹I, ³⁵S will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful.

It is understood that a “radio-labelled compound” is a compound that has incorporated at least one radionuclide. In some embodiments the radionuclide is selected from the group consisting of ³H, ¹⁴C, ¹²⁵I, ³⁵S and ⁸²Br.

Compounds of the present invention maybe administered orally, parenteral, buccal, vaginal, rectal, inhalation, insufflation, sublingually, intramuscularly, subcutaneously, topically, intranasally, intraperitoneally, intrathoracically, intravenously, epidurally, intrathecally, intracerebroventricularly and by injection into the joints.

The dosage will depend on the route of administration, the severity of the disease, age and weight of the patient and other factors normally considered by the attending physician, when determining the individual regimen and dosage level as the most appropriate for a particular patient.

The quantity of the compound to be administered will vary for the patient being treated and will vary from about 100 ng/kg of body weight to 100 mg/kg of body weight per day and preferably will be from 10 pg/kg to 10 mg/kg per day. For instance, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention.

Salts of the compounds of the invention are preferably physiologically well tolerated and non toxic. Many examples of salts are known to those skilled in the art. All such salts are within the scope of this invention, and references to compounds include the salt forms of the compounds.

Where the compounds contain an amine function, these may form quaternary ammonium salts, for example by reaction with an alkylating agent according to methods well known to the skilled person. Such quaternary ammonium compounds are within the scope of the invention.

Compounds containing an amine function may also form N-oxides. A reference herein to a compound that contains an amine function also includes the N-oxide.

Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle.

N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 6^{th} Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (MCPBA), for example, in an inert solvent such as dichloromethane.

Where the compounds contain chiral centres, all individual optical forms such as enantiomers, epimers and diastereoisomers, as well as racemic mixtures of the compounds are within the scope of the invention.

### BRIEF DESCRIPTION OF THE DRAWINGS

FIG. 1A shows Example 3 bound to the BACE active site at 1.83 Å resolution. 2Fo-Fc map contoured at 1.4 sigma.

FIG. 1B shows Example 3 bound to the BACE active site at 1.83 Å resolution. 2Fo-Fc map contoured at 1.4 sigma.

FIG. 2A shows Example 12 bound to the BACE active site at 1.85 Å resolution. 2Fo-Fc map contoured at 1.3 sigma.

FIG. 2B shows Example 12 bound to the BACE active site at 1.85 Å resolution. 2Fo-Fc map contoured at 1.3 sigma.

### PREPARATION OF COMPOUNDS

The compounds of the present invention can be prepared as a free base or a pharmaceutically acceptable salt thereof by the processes described below. Throughout the following description of such processes it is understood that, where appropriate, suitable protecting groups will be added to, and subsequently removed from the various reactants and intermediates in a manner that will be readily understood by one skilled in the art of organic synthesis. Conventional procedures for using such protecting groups as well as examples of suitable protecting groups are for example described in Protective Groups in Organic Synthesis by T. W. Greene, P. G. M Wutz, 3^{rd} Edition, Wiley-Interscience, New York, 1999. It is understood that microwaves can alternatively be used for the heating of reaction mixtures. Another aspect of the present invention provides a process for preparing a compound of formula (I), or a pharmaceutically acceptable salt thereof, wherein R⁷ and R¹⁰ are defined as aryl, heterocyclyl or heteroaryl, and R¹, R² and R³ are, unless specified otherwise, as defined in formula (I). Said process comprises of:

### (i) Formation of a Corresponding Compound of Formula (V):

A compound of formula (V) may be obtained as depicted in Scheme 1, for example by metallation or halogen metal exchange of a compound of formula (II), wherein G is either a hydrogen or a halogen respectively, to obtain an intermediate of formula (III), wherein M is a metal such as Zn or Mg and L is a ligand such as halogen (such as iodo or bromo) and n is between 0 and 6. The reaction may be run with an additive such as LiCl. The intermediate (III) is optionally not isolated but reacted further with a compound of formula (IV), wherein LG is either N(CH₃)(OCH₃), SCH₂CH₃ or halogen, such as chloro, or another suitable leaving group as described for example by R. K. Dieter, (Tetrahedron, 55 (1999) 4177-4236).

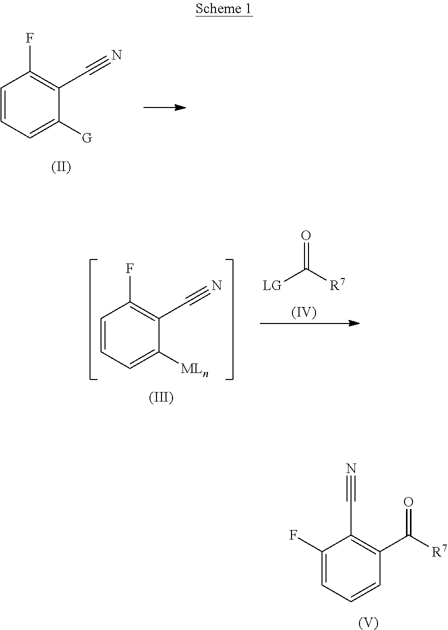

The reaction may be carried out by treating a compound of formula (II), wherein G is a halogen (such as iodine or bromine) with an appropriate metallating reagent, such as a lithium reagent (such as tert-butyllithium, n-butyllithium, lithium diispropylamide or lithium tetramethyl is piperidine) or with a Grignard reagent (such as isopropylmagnesium bromide) or with a metal (such as magnesium, zinc or manganese), by standard methods known in the art. Optionally, the formed intermediate of formula (III) may be further transmetallated by treatment with a metal salt or metal complex, such as copper cyanide di(lithium bromide), to obtain a new intermediate of formula (III), and then treat said intermediate of formula (III) with a compound of formula (IV), wherein LG represents a leaving group such as a halogen (such as chlorine) or N(CH₃)(OCH₃) or SCH₂CH₃. Optionally, this transformation may be performed under the influence of a transition metal catalyst, such as a copper salt, such as CuCN, or a palladium salt or complex, such as Pd(OAc)₂ or Pd(PPh₃)₄ optionally in the presence of an additional ligand, as described in the literature for example by R. K. Dieter, (Tetrahedron, 55 (1999) 4177-4236). The reaction may be performed in a suitable solvent, such as diethyl ether, tetrahydrofuran, 2-methyl-tetrahydrofuran, dimethylformamide, dichloromethane or acetonitrile, or mixtures thereof, at a temperature between −105° C. and room temperature.

The reagent (IV) in the case when LG is chloro might be generated in situ by for example reaction of the corresponding acid (VI) with a chlorinating agent such as oxalyl chloride (Scheme 2). Reagent (IV), when LG is chloro, can be reacted with ethanethiol to generate another compound (IV) wherein LG is SCH₂CH₃. The acid (VI) is commercially available or can be synthesised by methods known to a person skilled in the art of organic synthesis.

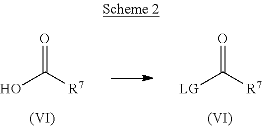

### (ii) Formation of a Corresponding Compound of Formula (IX):

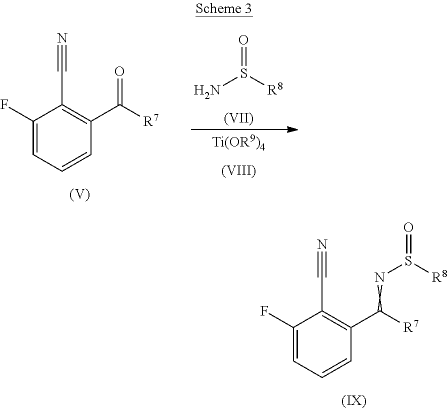

A compound of formula (IX) may be obtained by reacting a compound of formula (V) with a compound of formula (VII) (Scheme 3), wherein R⁸ is alkyl (such as for example tert-butyl). is Compound (VII) can be either a racemate or an enantiomerically enriched or enantiopure compound. The reaction is performed in the presence of a suitable Lewis acid, such as a titanium compound of formula (VIII), wherein R⁹ is alkyl (such as ethyl or isopropyl). The reaction is performed in a suitable solvent (such as diethyl ether, 2-methyl-tetrahydrofuran or tetrahydrofuran) at a temperature between room temperature and reflux temperature. If compound (VII) is an optically pure enantiomer the enantiomerically pure compound (IX) may be obtained.

(iii) Formation of a Corresponding Compound of Formula (XI)

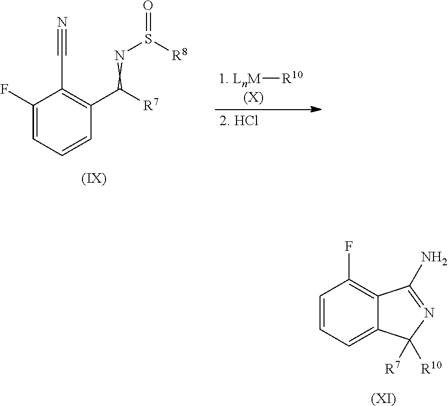

A compound of formula (XI) may be prepared as shown in Scheme 4 by treating a compound of formula (IX), with an appropriate organometallic reagent, or a mixture of organometallic reagents, of formula (X), wherein M is a metal (such as lithium, zinc or magnesium) L is a ligand (such as halogen) and n is between 0 and 2, followed by treatment with a suitable acid, such as hydrochloric acid. The reaction may be performed in a suitable solvent, such as diethyl ether, 2-methyl-tetrahydrofuran or tetrahydrofuran, at a temperature between −105° C. and room temperature. The organometallic reagent of formula (X) may be generated from the corresponding LG-R¹⁰, wherein LG represents a leaving group such as a halogen, (such as iodo, bromo or chloro), by known methods as described for example in Advanced Organic Chemistry by Jerry March 6^{th} edition, Wiley Interscience. If an enantiomerically pure, or enriched, compound (IX) is used in this reaction, an enantiomerically pure or enantiomerically enriched compound (XI) may be obtained.

### (iv) Formation of a Corresponding Compound of Formula (XIV)

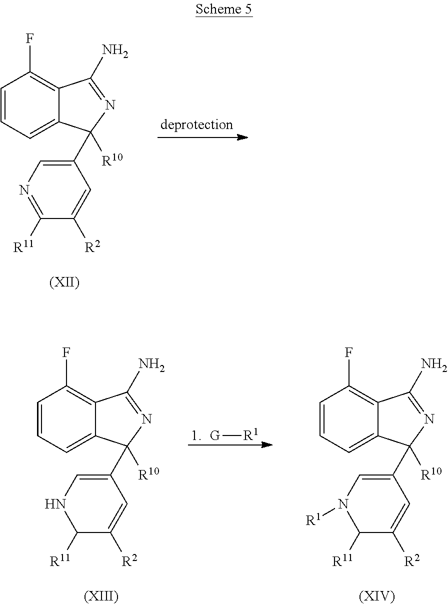

A compound of formula (XIV) may be prepared from a compound of formula (XII) wherein R¹¹ may be F, OCH₃ or OSiR′R″R′″ (wherein R′, R″ and R′″ are independently aryl (such as phenyl) or alkyl (such as methyl or tert-butyl)), as shown in Scheme 5. If R¹¹ is F the conversion into (XIII) may be carried out by for instance acidic hydrolysis using aqueous HCl. If R¹¹ is OCH₃ the conversion into (XIII) may be carried out by reaction with for instance TMSI in a suitable solvent such as CHCl₃ or by reaction with HBr in a suitable solvent such as acetic acid or by reaction with BBr₃ in a suitable solvent such as dichloromethane. If R¹¹ is OSiR′R″R′″ the conversion into (XIII) may be carried out by for instance HCl in a suitable solvent such as methanol or by using tetrabutyl ammonium fluoride in THF. Compound (XIV) may then be prepared from compound (XIII) using G-R¹ wherein G is a halogen (such as iodine or bromine) using an appropriate base (such as potassium carbonate or sodium hydride) in an appropriate solvent such as DMF or DME at a temperature between −20° C. and +40° C. If an enantiomerically pure or enriched compound (XII) is used in this reaction, an enantiomerically pure or enantiomerically enriched compound (XIV) may be obtained.

### (v) Formation of a Corresponding Compound of Formula (I):

A compound of formula (I) may be obtained (Scheme 6) by starting from, for example, a compound of formula (XV), wherein LG represents a leaving group such as halogen (such as chlorine, bromine or iodine), or an alkyl-, aryl- or haloalkyl-sulfonate (such as triflate), and reacting said compound (XV) with a compound of formula T-R³, wherein R³ is defined as above and T represents a boronic acid (B(OH)₂) or a boronic ester (B(OR)₂) or a stannane (SnR₃), under the influence of a transition metal catalyst as described in for example Metal-Catalyzed Cross-Coupling Reactions, 2nd, Completely Revised and Enlarged Edition by A de Meijere and F. Diederich, Wiley VCH, 2004. The compound of formula T-R³ may be generated from the corresponding LG-R³, wherein LG represents a leaving group, such as a halogen, (such as iodide, bromide or chlorine) by known methods as described in for example Advanced Organic Chemistry by Jerry March 6^{th} edition, Wiley Interscience.

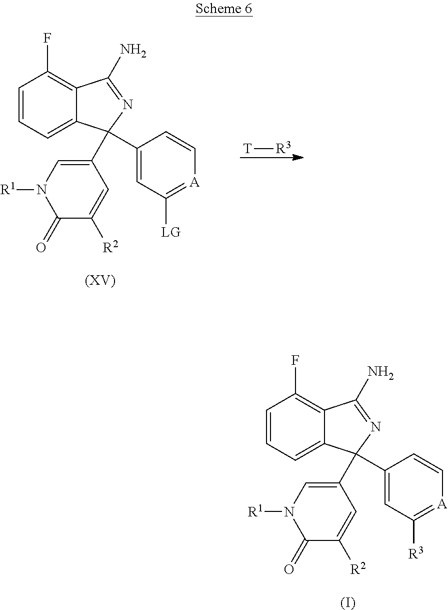

The reaction may be carried out by coupling of a compound of formula (XV), with an appropriate aryl or heteroaryl boronic acid or boronic ester or stannane of formula T-R³. The reaction may also be carried out using a suitable metal catalyst such as a palladium catalyst, such as di-tert-butylphosphinoferrocene palladium (II) dichloride, tetrakis(triphenylphosphine)-palladium(0), palladium diphenylphosphineferrocene dichloride, palladium(II) acetate or bis(dibenzylideneacetone) palladium(0). Optionally, a suitable ligand such as triphenylphosphine, tri-tert-butylphosphine or 2-(dicyclohexylphosphino)biphenyl is employed. A suitable base, such as cesium fluoride, an alkyl amine, such as triethyl amine, or an alkali metal or alkaline earth metal carbonate or hydroxide such as potassium carbonate, sodium carbonate, cesium carbonate, or sodium hydroxide, may be used in the reaction. Said reaction may be performed at a temperature range between +20° C. and +160° C., in a suitable solvent, such as toluene, tetrahydrofuran, 2-methyl-tetrahydrofuran, dioxane, dimethoxyethane, water, ethanol, N,N-dimethylacetamide or N,N-dimethylformamide, or mixtures thereof. If enantiomerically pure or enriched compound (XV) is used in this reaction, an enantiomerically pure or enantiomerically enriched compound (I) may be obtained.

Compounds of formula (II), (III), (IV), (VI), (VII), (VIII), (X), G-R¹ and T-R³ are commercially available compounds, or are known in the literature, or they are prepared by standard processes known in the art. A compound of formula (I), (XI), (XII), (XIII), (XIV) or (XV) may be separated into its enantiomers by standard processes known in the art by for example chromatography on a chiral stationary phase.

### General Methods

All solvents used were of analytical grade and commercially available anhydrous solvents were routinely used for reactions. Starting materials used were available from commercial sources, or prepared according to literature procedures. Room temperature refers to 20-25° C. Solvent mixture compositions are given as volume percentages or volume ratios. Microwave heating was performed in a Biotage Creator, Initiator or Smith Synthesizer Single-mode microwave cavity producing continuous irradiation at 2450 MHz. It is understood that microwaves can be used for the heating of reaction mixtures.

Thin layer chromatography (TLC) was performed on Merck TLC-plates (Silica gel 60 F₂₅₄) and spots were UV visualized. Straight phase flash column chromatography was manually performed on Merck Silica gel 60 (0.040-0.063 mm), or automatically using an ISCO Combiflash® Companion™ system using RediSep™ normal-phase flash columns using the solvent system indicated. Phase separation was optionally performed on an Isolute® phase is separator.

NMR spectra were recorded on a 400 MHz (or higher field) NMR spectrometer fitted with a probe of suitable configuration. Spectra were recorded at ambient temperature unless otherwise stated. Chemical shifts are given in ppm down- and upfield from TMS (0.00 ppm). The following reference signal was used: the residual solvent signal of DMSO-d_{6 δ} 2.5. Resonance multiplicities are denoted s, d, t, q, m, br and app for singlet, doublet, triplet, quartet, multiplet, broad and apparent, respectively.

### HPLC, HPLCMS and LCMS Analyses:

High pressure liquid chromatography (HPLC) was performed on a reversed phase (RP) column. A linear gradient was applied using for example mobile phase A (10 mM NH₄OAc in 5% CH₃OH or CH₃CN, or 0.1% NH₃ or 0.1% Formic Acid.) and B (CH₃OH or CH₃CN). Mass spectrometer (MS) analyses were performed in positive and/or negative ion mode using electrospray ionization (ESI+/−) and/or atmospheric pressure chemical ionization (APCI+/−).

### GCFID and GCMS Analyses:

Gas chromatography (GC) was performed on a GC equipped with a mass spectrometer (MS) or a flame ionization detector (FID). The MS ion source was either an electron impact (EI) or a chemical ionization (CI, reactant gas methane). For separation a capillary column was used for example DB-5MS, (J&W Scientific). A linear temperature gradient was applied.

Preparative chromatography was run on a Waters FractionLynx system with a Autosampler combined Automated Fraction Collector (Waters 2767), Gradient Pump (Waters 2525), Column Switch (Waters CFO) and PDA (Waters 2996). Column; XBridge® Prep C8 10 μm OBD™ 19×300 mm, with guard column; XTerra® Prep MS C8 10 μm 19×10 mm Cartridge. A gradient of A (95% 0.1 M NH₄OAc in MilliQ water and 5% MeCN) in B (100% MeCN) or a gradient of A (95% 0.1 M NH₄OAc in MilliQ water and 5% MeOH), A (0.2% NH₃ in MilliQ water) or A (0.2% formic acid in MilliQ water) in B (100% MeOH) was applied for LC-separation at flow rate 20 ml/min.

Preparative chiral chromatography for separation of enantiomers was run on an LaPrep® system using the specified column and mobile phase system.

Compounds have been named using CambridgeSoft MedChem ELN v2.2.

### Abbreviations

aq. aqueous
DCM dichloromethane
DEA diethylamine
DME 1,2-dimethoxyethane
DMF N,N-dimethyl formamide
DMSO dimethyl sulfoxide
Et₂O diethyl ether
EtOAc ethyl acetate
EtOH ethanol
h hour(s)
HPLC high performance (or pressure) liquid chromatography
LCMS liquid chromatography mass spectrometry
MeOH methanol
min minute(s).
MS mass spectrometry
NH₄OAc ammonium acetate
NMR nuclear magnetic resonance
quant. quantitative
r.t. room temperature
sat. saturated
TFA trifluoroacetic acid
THF tetrahydrofuran

### Examples

Below follows a number of non-limiting examples of compounds of the invention.

### Example 1i

### (S)-N-((3-Bromophenyl)(2-cyano-3-fluorophenyl)methylene)-2-methylpropane-2-sulfinamide

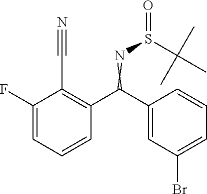

Titanium(IV) ethoxide (110 mL, 526 mmol) was added to 2-(3-bromobenzoyl)-6-fluorobenzo-nitrile (64 g, 210.45 mmol, WO2010/056196) in 2-methyl-tetrahydrofuran (500 mL) under argon at r.t. After 5 min (S)-2-methylpropane-2-sulfinamide (28.1 g, 231 mmol) was added in one portion. After 18 h the reaction was cooled to r.t. and MeOH (75 mL), sat. NaHCO₃ (225 mL) and EtOAc (500 mL) were added. The mixture was stirred for 10 min, and was allowed to stand for 30 min before it was decanted. EtOAc (2×500 mL) was added and stirred for 10 min after which it was decanted. The combined organic phases were washed with water (400 mL) dried (Mg2SO₄), filtered and concentrated. After drying under vacuum the crude material was slurried in n-heptane:EtOAc 3:1 (200 mL). The mixture was stirred overnight and then it was filtered.

Drying under vacuum overnight gave the title compound (48.6 g, 57% yield). The mother liquor was purified by silica gel column chromatography using a gradient of heptane to heptane:EtOAc 1:1 affording additional title compound (24.4 g, 29% yield). MS (ES+) m/z 407, 409 [M+H]⁺.

### Example 2i

### (R)-5-(3-Amino-1-(3-bromophenyl)-4-fluoro-1H-isoindol-1-yl)-1-ethyl-3-methylpyridin-2(1H)-one

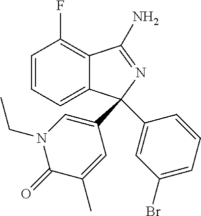

To a dry reactor was added n-butyllithium (53.4 mL, 133 mmol) and THF (100 mL). After cooling the mixture to inner temperature −25° C. was added n-butyl magnesium chloride (39.0 mL, 66.71 mmol) during 20 min. After 45 min. 5-bromo-1-ethyl-3-methylpyridin-2(1H)-one (39.9 g, 185 mmol, M. Ando et al. Bioorganic & Medicinal Chemistry 17 (2009) pp 6106-6122) in THF (100 mL) was added during 30 min. After 30 min. (S)—N-((3-bromophenyl)(2-cyano-3-fluorophenyl)methylene)-2-methylpropane-2-sulfinamide (41.8 g, 103 mmol, Example 11) dissolved in THF (100 mL) was added during 30 min. The mixture was allowed to reach r.t. during 45 min. The mixture was stirred at r.t. for 2 h. After cooling the mixture to inner temperature −20° C. ethylenediaminetetraacetic acid (1.42 g) was added followed by a mixture consisting of ammonium chloride (25.6 g) and water (150 mL) during 20 minutes, keeping the inner temperature <0° C. during the quench. The mixture was allowed to attain r.t. over 50 min. To the mixture was added isopropyl acetate (350 mL) and stirred for 15 min. The water phase was taken out from the reactor and NaCl (20 g) in water (100 mL) was charged to the organic phase and stirred for 5 min. The water phase was combined with the water phase above. The combined water phases were charged with isopropylacetate (150 mL) and stirred for 5 minutes after which the phases were separated. NaCl (20 g) in water (100 mL) was charged to the organic phase and stirred for 5 min and the phases were separated. Ethylenediaminetetraacetic acid (1 g) and 2 M citric acid (300 mL) were added to the combined organic phases (fraction 1) and the mixture was stirred for 45 min. The phases were separated and the water phase was transferred back to the reactor and isopropylacetate (200 mL) was added. The pH was adjusted to ˜12 using 4 M NaOH and the mixture was stirred for 10 min before MeOH (50 mL) was added. The phases were separated and the water phase was extracted by DCM (200 mL). The DCM and the isopropylacetate phases were combined, dried (Mg₂SO₄), filtered, concentrated and dried under vacuum to give the title compound with an enantiomeric purity of 73%. (24 g, 53% yield).

To fraction 1 was added HCl in MeOH (1.25 M, 50 mL) and the mixture was stirred for 2 h. Citric acid (2 M, 100 mL) was added and the mixture was stirred for 15 min before the phases were separated. To the water phase was added DCM and the pH was adjusted to ˜10 using 4 M NaOH. The collected organic phases were concentrated and dried under vacuum overnight. To the crude material was EtOAc (100 mL) added and the mixture was heated to boiling and then allowed cool to r.t. The formed material was filtered and dried overnight under vacuum to give the title compound with an enantiomeric purity of 55% (5.2 g, 12% yield). The two batches of the title compound were injected on a Chiralpak AD-H column (50×300 mm) using 80% n-heptane/20% EtOH (containing 0.1% DEA) as eluent (flowrate 120 mL/min). Detection was monitored at 254 nm. The second eluting enantiomer (retention time 11.5 min), (IIg, 99.8% enantiomeric purity) was collected: ¹H NMR (600 MHz, DMSO-d₆) δ ppm 1.14 (t, 3H), 1.94 (s, 3H), 3.83 (m, 2H), 6.60 (br. s., 2H), 7.25 (m, 4H), 7.33 (d, 1H), 7.42 (m, 2H), 7.52 (td, 1H), 7.60 (d, 1H); MS (ES+) m/z 440, 442 [M+H]⁺.

### Example 3i

### 4-Fluoro-3-methyl-5-(tributylstannyl)pyridine

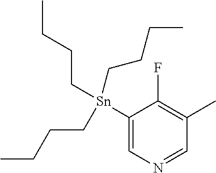

To a solution of lithium diisopropylamide (1.8 M in THF/heptane/ethylbenzene) (6.0 mL, 10.8 mmol) in dry THF (25.0 mL) at −78° C. under argon was 4-fluoro-3-methylpyridine (1.00 g, 9.00 mmol) added over 1 min. The resulting solution was stirred for 35 min, then tri-n-butyltin chloride (2.69 mL, 9.90 mmol) was added over 2 min. The mixture was stirred for 2 h at −78° C., then allowed to reach room temperature. The reaction was quenched by the addition of methanol, followed by concentration in vacuo. The residue was partitioned between brine and dichloromethane (×2). The combined organic layers were passed through a phase separator and concentrated. Purification by silica gel chromatography using a gradient of 0% to 5% methanol in dichloromethane gave the title compound (1.48 g, 41% yield): ¹H NMR (400 MHz, DMSO-d₆) δ ppm 0.78-0.90 (m, 10H), 1.09-1.16 (m, 5H), 1.28 (m, 7H), 1.44-1.55 (m, 5H), 2.20 (s, 3H), 8.31 (m, 1H), 8.41 (d, 1H); MS (ES+) m/z 402 [M+H]⁺.

### Example 4i

### 5-(3-Amino-1-(3-bromophenyl)-4-fluoro-1H-isoindol-1-yl)-1-isopropyl-3-methylpyridin-2(1H)-one

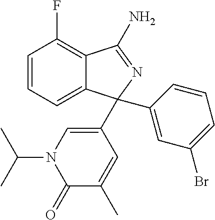

5-(3-Amino-1-(3-bromophenyl)-4-fluoro-1H-isoindol-1-yl)-3-methylpyridin-2(1H)-one (500 mg, 1.21 mmol, example 13i) was added to a suspension of sodium hydride (53 mg, 1.33 mmol) in N,N-dimethylformamide (8 mL) under argon. The mixture was stirred for 10 min. 2-Iodopropane (0.169 mL, 1.70 mmol) was added and the mixture was stirred at r.t. for two days. Another portion of 2-iodopropane (0.048 mL, 0.49 mmol) was added and the stirring was continued for two h. The reaction mixture was quenched by the addition of 4 M NH₄OH (0.5 mL). After 12 h the mixture was diluted with dichloromethane (20 mL), sat. aq. NaHCO₃ (20 mL) and EtOAc (20 mL). The layers was separated and the aqueous layer was extracted with EtOAc (20 mL). The combined organics were dried (Mg₂SO₄), filtered and concentrated. Purification by flash silica gel chromatography using a gradient of 100% A to 80% B in A (A: dichloromethane, B: 0.1 N NH₃ in 10% MeOH in dichloromethane) gave the title compound (164 mg, 30% yield): ¹H NMR (400 MHz, DMSO-d₆) δ ppm 1.13 (d, 3H), 1.20 (d, 3H), 1.93 (s, 3H), 4.96 (app. septet, 1H), 6.62 (br. s., 2H), 7.17-7.34 (m, 5H), 7.39 (t, 1H), 7.42 (m, 1H), 7.53 (td, 1H), 7.61 (m, 1H); MS (ES+) m/z 454, 456 [M+H]⁺.

### Example 5i

### tert-Butyl 1-(3-bromophenyl)-4-fluoro-1-(1-methyl-6-oxo-5-(trifluoromethyl)-1,6-dihydropyridin-3-yl)-1H-isoindol-3-ylcarbamate

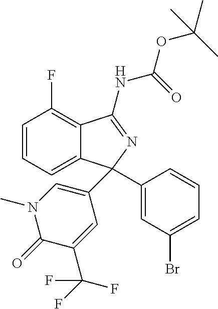

Methyl iodide (0.031 mL, 0.49 mmol) was added to a mixture of tert-butyl 1-(3-bromophenyl)-4-fluoro-1-(6-oxo-5-(trifluoromethyl)-1,6-dihydropyridin-3-yl)-1H-isoindol-3-ylcarbamate (232 mg, 0.41 mmol, Example 61) and potassium carbonate (85 mg, 0.62 mmol) in DME (5 mL) at r.t. under a nitrogen atmosphere. The resulting mixture was stirred at 50° C. for 1 h. After cooling to r.t. the mixture was diluted with DCM (5 mL) then poured into a phase separator. The organic phase was collected and concentrated to give the title compound (260 mg, quant. yield). MS (ES+) m/z 580, 582 [M+H]⁺.

### Example 6i

### tert-Butyl 1-(3-bromophenyl)-4-fluoro-1-(6-oxo-5-(trifluoromethyl)-1,6-dihydropyridin-3-yl)-1H-isoindol-3-ylcarbamate

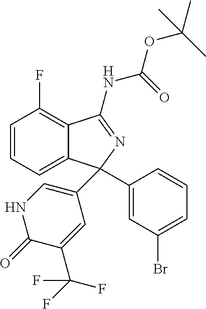

5-(3-Amino-1-(3-bromophenyl)-4-fluoro-1H-isoindol-1-yl)-3-(trifluoromethyl)pyridin-2(1H)-one (190 mg, 0.41 mmol, Example 71), di-tert-butyl dicarbonate (0.103 mL, 0.45 mmol) and 4-dimethylaminopyridine (4.98 mg, 0.04 mmol) were dissolved in THF (5 mL) and stirred at r.t. under a nitrogen atmosphere overnight. The mixture was diluted with DCM (6 mL) and sat. aq. NaHCO₃ (6 mL) and then poured into a phase separator. The organic phase was collected and concentrated to give the title compound (245 mg, quant. yield). MS (ES+) m/z 566, 568 [M+H]⁺.

### Example 7i

### 5-(3-Amino-1-(3-bromophenyl)-4-fluoro-1H-isoindol-1-yl)-3-(trifluoromethyl)pyridin-2(1H)-one

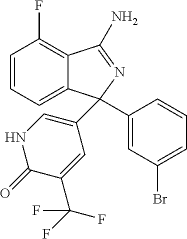

n-Butyllithium (9.60 mL, 24.00 mmol) was added dropwise to a mixture of 2-(tert-butyldimethylsilyloxy)-5-iodo-3-(trifluoromethyl)pyridine (8.95 g, 22.2 mmol, Example 81) in THF (60 mL) at −78° C. under a nitrogen atmosphere. The resulting mixture was stirred at −78° C. for 5 min, then a mixture of N-((3-bromophenyl)(2-cyano-3-fluorophenyl)methylene)-2-methylpropane-2-sulfinamide (8.15 g, 20 mmol, WO2010/056196) in THF (30 mL) was added over 10 min. The mixture was stirred at −78° C. for 15 min then the cooling bath was removed and the mixture was stirred at r.t. for 3.5 h. Hydrogen chloride—1.25 M methanol solution (48.0 mL, 60.0 mmol) was added and the mixture was stirred at r.t. for 15 min. The volatiles were removed in vacuo and the resulting residue was dissolved in DCM (50 mL) and sat. aq. NaHCO₃ (50 mL). The mixture was poured into a phase separator, the organic phase was collected, concentrated and purified on a silica gel column using a gradient of 0-5% 0.1 M NH₃ in MeOH is in DCM. The residue was re-dissolved in DCM and then 1 M HCl in Et₂O (19 mL) was added. The resulting mixture was stirred for 15 min at r.t. The formed precipitate was collected and washed with DCM then taken up in DCM and sat. aq. NaHCO₃. The mixture was poured into a phase separator, the organic phase was collected and concentrated to give the title compound (4.60 g, 49% yield). ¹H NMR (400 MHz, DMSO-d₆) δ ppm 6.74 (br. s., 2H), 7.24-7.34 (m, 3 H), 7.40 (dt, 1H), 7.43-7.51 (m, 2H), 7.54 (td, 1H), 7.63 (d, 1H), 7.68 (d, 1H), 12.21 (br. s., 1 H); MS (ES+) m/z 466, 468 [M+H]⁺.

### Example 8i

### 2-(tert-Butyldimethylsilyloxy)-5-iodo-3-(trifluoromethyl)pyridine

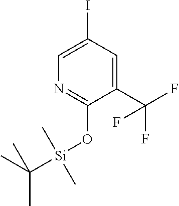

Triethylamine (6.52 mL, 46.8 mmol) was added to a mixture of tert-butyldimethylchlorosilane (4.98 mL, 26.7 mmol) and 5-iodo-3-(trifluoromethyl)pyridin-2(1H)-one (6.44 g, 22.3 mmol) in DCM (50 mL) at r.t. After 4 h the mixture was diluted with water (20 mL), poured into a phase separator, the organic phase was collected and concentrated to give the title compound (9.10 g, quant. yield). ¹H NMR (400 MHz, DMSO-d₆) δ ppm 0.32 (s, 6H), 0.95 (s, 9H), 8.35 (d, 1H), 8.62 (d, 1H); MS (CI-) m/z 404 [M+H]⁺.

### Example 9i

### 5-(3-Amino-1-(3-bromophenyl)-4-fluoro-1H-isoindol-1-yl)-1,3-dimethylpyridin-2(1H)-one

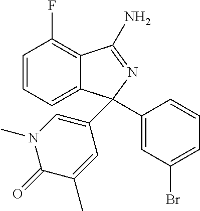

Method A: tert-Butyllithium (1.969 mL, 3.15 mmol) was added dropwise to a solution of 5-bromo-1,3-dimethylpyridin-2(1H)-one (333 mg, 1.65 mmol, C. Morrow et al. J. Org. Chem., 1974, 39 (14), pp 2116-2118) in THF (1.5 mL) at −100° C. under a nitrogen atmosphere. After 5 min a solution of N-((3-bromophenyl)(2-cyano-3-fluorophenyl)methylene)-2-methylpropane-2-sulfinamide (611 mg, 1.5 mmol, WO2010/056196) in THF (2 mL) was added. After 30 min on the thawing cooling bath and 1 h at rt hydrogen chloride—1.25 M methanol solution (4.80 mL, 6.00 mmol) was added and the resulting mixture was stirred at rt for 1 h. The mixture was concentrated and the resulting residue was dissolved in DCM and sat. aq. NaHCO₃. The mixture was poured into a phase separator, the organic phase was concentrated and purified on a silica gel column eluted with a gradient of 0-8% 0.1M NH₃ in MeOH in DCM to give the title compound (110 mg, 17% yield). ¹H NMR (400 MHz, DMSO-d₆) δ ppm 1.94 (s, 3H), 3.35 (s, 3H), 6.60 (br. s., 2H), 7.20-7.30 (m, 4H), 7.34 (dt, 1H), 7.43 (tt, 2H), 7.52 (td, 1H), 7.59 (d, 1H); MS (ES+) m/z 426, 428 [M+H]⁺.

### Method B:

5-(3-Amino-1-(3-bromophenyl)-4-fluoro-1H-isoindol-1-yl)-3-methylpyridin-2(1H)-one (1.497 g, 3.63 mmol, Example 13i) was added to a suspension of sodium hydride (0.189 g, 4.72 mmol) in DMF (37 mL). The mixture was tirred at r.t. for 4 min then was methyl iodide (0.294 mL, 4.72 mmol) added. After 1.5 h it was combined with a similar reaction based on 100 mg (0.24 mmol) 5-(3-amino-1-(3-bromophenyl)-4-fluoro-1H-isoindol-1-yl)-3-methylpyridin-2(1H)-one. The reaction mixture was quenched with water and the pH was adjusted to approximately 8 using HCl (1 M). NaHCO₃ (aq., sat) was added and the mixture was extracted with CH₂Cl₂ (×2) and EtOAc (×3). The combined organics were dried (Na₂SO₄), filtered, concentrated and co-evaporated once with toluene to give the crude title compound (1.887 g, quant. yield). ¹H NMR (500 MHz, DMSO-d₆) δ ppm 1.94 (s, 3H), 3.36 (s, 3H), 6.57 (br. s., 2H), 7.22-7.29 is (m, 4H), 7.34 (d, 1H), 7.40-7.45 (m, 2H), 7.52 (td, 1H), 7.58 (m, 1H); MS (ES+) m/z 426, 428 [M+H]⁺.

### Example 10i

### 5-(3-Amino-1-(2-bromopyridin-4-yl)-4-fluoro-1H-isoindol-1-yl)-1,3-dimethylpyridin-2(1H)-one

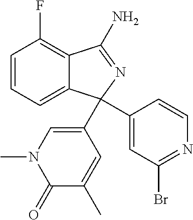

### Method A

n-Butyllithium (0.229 mL, 0.57 mmol) was added to 5-bromo-1,3-dimethylpyridin-2(1H)-one (116 mg, 0.57 mmol, C. Morrow et al. J. Org. Chem., 1974, 39 (14), pp 2116-2118) in THF (7 mL) at −78° C. under nitrogen atmosphere. After 30 min N-((2-bromopyridin-4-yl)(2-cyano-3-fluorophenyl)methylene)-2-methylpropane-2-sulfinamide (180 mg, 0.44 mmol, WO2010/056196) in THF (2 mL) was added. After 1 h at −78° C. methanol (5 mL) was added followed by hydrochloric acid in diethylether (1.32 mL, 1.32 mmol) and the reaction was stirred for 1 hour at room temp. The reaction was quenched with NaHCO₃ (aq.sat.) and extracted with EtOAc. The solvent was removed in vacuo to give the crude title compound (190 mg) that was used as such in the next step. MS (ES+) m/z 427 [M+H]⁺.

### Method B

5-Bromo-1,3-dimethylpyridin-2(1H)-one (143 mg, 0.71 mmol, C. Morrow et al. J. Org. Chem., 1974, 39 (14), pp 2116-2118) was stirred under Ar(g) for 3 min and dissolved in THF (2 mL). Isopropylmagnesiumchloride lithiumchloride complex (1.3 M in THF) (0.544 mL, 0.71 mmol) was added dropwise and the mixture were stirred for 10 min. N-((2-bromopyridin-4-yl)(2-cyano-3-fluorophenyl)methylene)-2-methylpropane-2-sulfinamide (170 mg, 0.42 mmol, WO2010/056196) in THF (2 mL) was added. The mixture was stirred for 2 h. HCl in MeOH (1.25 M, 2.33 mL, 2.91 mmol) was added and the mixture were stirred another h. NaHCO₃(aq. sat) was added and the mixture was extracted with EtOAc. The organic phases were pooled, dried over MgSO₄ and concentrated. The crude product was purified by silica gel chromatography using a gradient of 0-4% MeOH(containing NH₃) in DCM to give the title is compound (140 mg, 79% yield): ¹H NMR (500 MHz, DMSO-d₆) δ ppm 1.94 (s, 3H), 3.36 (s, 3 H), 6.71 (br. s., 2H), 7.23 (m, 1H), 7.29 (m, 2H), 7.40 (m, 1H), 7.46 (m, 1H), 7.56 (m, 1H), 7.65 (m, 1H), 8.30 (d, 1H); MS (ES+) m/z 427 [M+H]⁺.

### Example 11i

### 5-(3-Amino-1-(2-bromopyridin-4-yl)-4-fluoro-1H-isoindol-1-yl)-1,3-diethylpyridin-2(1H)-one

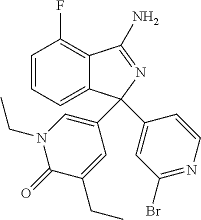

Sodium hydride (35 mg, 0.88 mmol) in dry N,N-dimethylformamide (15.0 mL) was stirred for 5 minutes before the addition of 5-(3-amino-1-(2-bromopyridin-4-yl)-4-fluoro-1H-isoindol-1-yl)-3-ethylpyridin-2(1H)-one (290 mg, 0.68 mmol, example 12i). After 20 min the mixture was cooled to 0° C. (ice-water bath) and iodoethane (0.213 mL, 2.67 mmol) was added dropwise over 1 min. After 2 h at 0° C. the reaction was quenched by the addition of water (2 mL). After 40 min the resulting mixture was partitioned between aq. sodium bicarbonate (sat.) and ethyl acetate and the mixture was extracted with ethyl acetate (×3). The combined organic layers were dried (Na₂SO₄), filtered and concentrated. Purification by silica gel chromatography using a gradient of n-heptane/EtOAc gave the title compound (140 mg, 45% yield). ¹H NMR (500 MHz, DMSO-d₆) δ ppm 0.99 (t, 3H), 1.14 (t, 3H), 2.36 (q, 2H), 3.75-3.91 (m, 2H), 6.74 (br. s., 2H), 7.14 (d, 1H), 7.24-7.34 (m, 2H), 7.40 (dd, 1H), 7.45 (d, 1H), 7.56 (td, 1H), 7.66 (d, 1H), 8.31 (d, 1H); MS (ES+) m/z 455, 457 [M+H]⁺.

### Example 12i

### 5-(3-Amino-1-(2-bromopyridin-4-yl)-4-fluoro-1H-isoindol-1-yl)-3-ethylpyridin-2(1H)-one

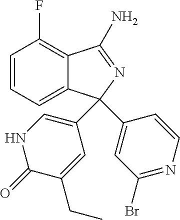

n-Butyllithium (2.5 M in hexane) (1.176 mL, 2.94 mmol) was added over 10 min to 5-bromo-2-(tert-butyldimethylsilyloxy)-3-ethylpyridine (0.852 g, 2.69 mmol, Example 17i) in dry THF (5 mL) at −78° C. under argon. After 25 min N-((2-bromopyridin-4-yl)(2-cyano-3-fluorophenyl)-methylene)-2-methylpropane-2-sulfinamide (1 g, 2.45 mmol, WO2010/056196) in THF (15 mL) was added over 35 min. The reaction was kept at −78° C. for 2 h and then hydrochloric acid (1.25 is M in methanol) (5.88 mL, 7.35 mmol) was added and the mixture was stirred at r.t. for 1 h. Water was added and the pH was adjusted to 8 with NaOH (10%, aq.). The mixture was extracted with EtOAc (×3), dried (Na₂SO₄), filtered and concentrated. Purification by silica gel chromatography using a gradient of 0% to 10% (3.5 M ammonia in methanol) in DCM gave the title compound (0.280 g, 27% yield). ¹H NMR (500 MHz, DMSO-d₆) δ ppm 0.98 (t, 3H), 2.32 (q, 2H), 6.74 (br. s., 1H), 6.90 (d, 1H), 7.16 (d, 1H), 7.29 (t, 1H), 7.41 (dd, 1H), 7.47 (s, 1H), 7.54 (td, 1H), 7.63 (d, 1H), 8.30 (d, 1H), 11.33 (br. s., 1H); MS (ES+) m/z 427, 429 [M+H]⁺.

### Example 13i

### 5-(3-Amino-1-(3-bromophenyl)-4-fluoro-1H-isoindol-1-yl)-3-methylpyridin-2(1H)-one

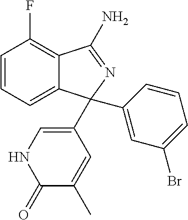

n-Butyllithium (1.497 mL, 3.74 mmol) was added dropwise over 3 min to a solution of 5-bromo-2-(tert-butyldimethylsilyloxy)-3-methylpyridine (0.943 g, 3.12 mmol, Example 14i) in dry THF (10 mL) at −70° C. under Ar (g). After 35 min a solution of N-((3-bromophenyl)(2-cyano-3-fluorophenyl)methylene)-2-methylpropane-2-sulfinamide (1.27 g, 3.12 mmol, WO2010/056196) in dry THF (5 mL) was added dropwise over 3 min. After 25 min the flask was taken up from the cooling bath. After a further 35 min hydrogen chloride-1.25 M methanol solution (7.48 mL, 9.35 mmol) was added. After 40 min water was added and the pH was adjusted to 8 with NaOH (10%, aq.). The mixture was extracted with EtOAc (×3), dried (Na₂SO₄), filtered and concentrated to give the crude title compound (1.58 g, quant. yield). ¹H NMR (600 MHz, DMSO-d₆) δ ppm 1.90 (s, 3H), 6.58 (br. s., 2H), 6.87 (d, 1H), 7.21 (dd, 1H), 7.23-7.29 (m, 2 H), 7.35 (m, 1H), 7.40-7.46 (m, 2H), 7.51 (td, 1H), 7.55 (d, 1H), 11.25 (br. s., 1H); MS (ES+) m/z 412, 414 [M+H]⁺.

### Example 14i

### 5-Bromo-2-(tert-butyldimethylsilyloxy)-3-methylpyridine

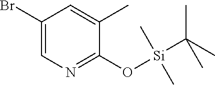

Triethylamine (29.4 mL, 211 mmol) was added to a suspension of 5-bromo-2-hydroxy-3-methylpyridine (15.9 g, 84.6 mmol) and tert-butyldimethylchlorosilane (15.29 g, 101 mmol) in CH₂Cl₂ (280 mL). After 4 h at r.t. water was added and the phases were separated. The organic phase was washed once with water, dried (Na₂SO₄), filtered, concentrated and co-evaporated twice with toluene to give the title compound (25.8 g, quant. yield): ¹H NMR (400 MHz, DMSO-d₆) δ ppm 0.26-0.29 (m, 6H), 0.96 (s, 9H), 2.12 (s, 3H), 7.80 (dd, 1H), 8.03 (d, 1H); MS (ES+) m/z 302, 304 [M+H]⁺.

### Example 15i

### 5-(3-Amino-1-(3-bromophenyl)-4-fluoro-1H-isoindol-1-yl)-1-ethyl-3-methylpyridin-2(1H)-one

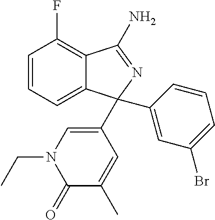

Sodium hydride (0.164 g, 4.10 mmol) was stirred at room temperature with N,N-dimethylformamide (25.0 mL) for 5 minutes before the addition of 5-(3-amino-1-(3-bromophenyl)-4-fluoro-1H-isoindol-1-yl)-3-methylpyridin-2(1H)-one (1.47 g, 3.57 mmol, Example 13i). The mixture was stirred for 20 min. at r.t., cooled to 0° C. and iodoethane (0.432 mL, 5.35 mmol) was added dropwise over 1 min. The reaction was stirred at 0° C. for 2 h and was then quenched by the addition of water (2 mL). After 40 min the cooling bath was removed. The reaction mixture was partitioned between sodium bicarbonate (aq. sat.) and ethyl acetate (x 3). NaCl (s) was added to the water phase and it was extracted with ethyl acetate (×4). The combined organic layers were dried (Na₂SO₄), filtered and concentrated in vacuo. Purification by silica gel chromatography using a gradient of 0% to 10% (3.5 M ammonia in methanol) in dichloromethane gave the title compound (1.16 g, 74% yield). ¹H NMR (500 MHz, DMSO-d₆) d ppm 1.14 (t, 3H), 1.94 (s, 3H), 3.83 (m, 2H), 6.60 (br. s., 2H), 7.21-7.29 (m, 4H), 7.33 (m, 1 H), 7.40-7.45 (m, 2H), 7.52 (td, 1H), 7.60 (d, 1H); MS (ES+) m/z 440, 442 [M+H]⁺.

### Example 16i

### 5-Bromo-3-ethylpyridin-2-ol

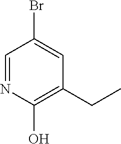

A mixture of 3-ethylpyridin-2-ol (1.05 g, 8.53 mmol) and N-bromosuccinimide (1.517 g, 8.53 mmol) in dry acetonitrile (40 mL) was stirred in the dark under a nitrogen atmosphere at r.t. overnight. The mixture was concentrated and the resulting residue was taken up in CCl₄ (30 mL), the precipitate was filtered off and the filtrate was concentrated to give the title compound (1.70 g, 99% yield). ¹H NMR (500 MHz, DMSO-d₆) δ ppm 1.07 (t, 3H), 2.37 (q, 2H), 7.36 (m, 1H), 7.49 (m, 1H), 11.74 (br. s., 1H); MS (ES+) m/z 202, 204 [M+H]⁺.

### Example 17i

### 5-Bromo-2-(tert-butyldimethylsilyloxy)-3-ethylpyridine

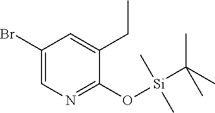

Triethylamine (0.860 mL, 6.19 mmol) was added to a solution of 5-bromo-3-ethylpyridin-2-ol (500 mg, 2.47 mmol, Example 16i) and tert-butyldimethylchlorosilane (448 mg, 2.97 mmol) in CH₂Cl₂ (8 mL). After 3 h water was added and the phases were separated. The organic phase was washed with water once, dried (Na₂SO₄), filtered, concentrated and co-evaporated twice with toluene to give the title compound (775 mg, 99% yield): ¹H NMR (500 MHz, DMSO-d₆) δ ppm 0.28 (s, 6H), 0.97 (s, 9H), 1.12 (t, 3H), 2.48-2.55 (m, 2H), 7.76 (d, 1H), 8.04 (d, 1H); MS (ES+) m/z 316, 318 [M+H]⁺.

### Example 18i

### (R)-5-(3-Amino-1-(3-bromophenyl)-4-fluoro-1H-isoindol-1-yl)-3-methylpyridin-2(1H)-one

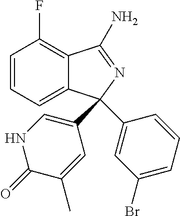

n-Butyllithium (2.5 M in hexane) (9.43 mL, 23.57 mmol) was added over 10 min to 5-bromo-2-(tert-butyldimethylsilyloxy)-3-methylpyridine (6.53 g, 21.6 mmol, Example 14i) in THF (30 mL) at −78° C. under argon atmosphere. The reaction was stirred for 25 min, then (S)—N-((3-bromophenyl)(2-cyano-3-fluorophenyl)methylene)-2-methylpropane-2-sulfinamide (8 g, 19.64 mmol, Example 11) in THF (15 mL) was added over 35 min. The reaction was kept at −78° C. for 2 hours and then hydrochloric acid (1.25 M in methanol) (47.1 mL, 58.92 mmol) was added to the mixture and stirred at rt for 1 h. To the mixture was added water and the pH was adjusted to 8 with NaOH (10%, aq.), extracted with EtOAc (×3), dried (Na₂SO₄), filtered and concentrated. Purification by silica gel chromatography using 0% to 10% (3.5 M ammonia in methanol) in dichloromethane gave the title compound as an unknown mixture with the other enantiomer (5.05 g, 63% yield). MS (ES+) m/z 412, 414 [M+H]⁺.

### Example 19i

### (R)-5-(3-Amino-1-(3-bromophenyl)-4-fluoro-1H-isoindol-1-yl)-1,3-dimethylpyridin-2(1H)-one

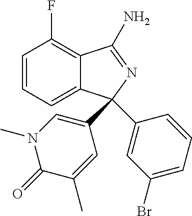

Sodium hydride (0.112 g, 2.79 mmol) was stirred at room temperature with N,N-dimethyl-formamide (15 mL) for 5 minutes before the addition of (R)-5-(3-amino-1-(3-bromophenyl)-4-fluoro-1H-isoindol-1-yl)-3-methylpyridin-2(1H)-one (1 g, 2.43 mmol, Example 18i) in one portion was made. The mixture was stirred for 20 minutes at room temperature, cooled to 0° C. (ice-water bath) and iodomethane (0.227 mL, 3.64 mmol) was added dropwise over 1 minute. The reaction was stirred at 0° C. for 2 h and then quenched by the addition of water (2 mL). The mixture was stirred at 0° C. for 40 min and the cooling bath was removed. The reaction mixture was partitioned between NaHCO₃ (aq.sat.) and EtOAc (×3). NaCl was added to the water phase and it was extracted with EtOAc (×4). The combined organic layers were dried (Na₂SO₄), filtered and concentrated in vacuo. Purification by preparative HPLC gave the title compound as an unknown mixture with the other enantiomer (0.150 g, 15% yield). ¹H NMR (500 MHz, is DMSO-d₆) δ ppm 1.94 (s, 3H), 3.36 (s, 3H), 6.59 (br. s., 2H), 7.19-7.30 (m, 4H), 7.34 (m, 1 H), 7.39-7.45 (m, 2H), 7.52 (m, 1H), 7.59 (m, 1H); MS (ES+) m/z 426, 428 [M+H]⁺.

### Example 20i

### 5-(3-Amino-1-(2-bromopyridin-4-yl)-4-fluoro-1H-isoindol-1-yl)-1-ethyl-3-methylpyridin-2(1H)-one

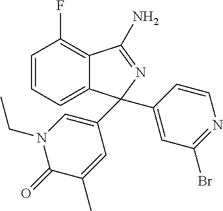

5-Bromo-1-ethyl-3-methylpyridin-2(1H)-one (236 mg, 1.09 mmol, M. Ando et al. Bioorg. Med. Chem.17 (2009) pp 6106-6122) was stirred under Ar(g) for 3 min and dissolved in THF (2.5 mL). Isopropylmagnesiumchloride lithiumchloride complex (1.3 M in THF) (0.817 mL, 1.06 mmol) was added dropwise and the mixture was stirred for 10 min. N-((2-bromopyridin-4-yl)(2-cyano-3-fluorophenyl)methylene)-2-methylpropane-2-sulfinamide (255 mg, 0.62 mmol, WO2010/056196) in THF (2.5 mL) was added. The mixture were stirred for 2 h. HCl in MeOH 1.25 M (3.00 mL, 3.75 mmol) was added and the mixture was stirred another 2 h. NaHCO₃ (sat) was added and the mixture was extracted with EtOAc. The organic phases were pooled, dried over MgSO₄ and concentrated. Silica gel column chromatography using a gradient of 0-6% MeOH (containing NH₃) in DCM gave the title compound (230 mg, 83% yield). ¹H NMR (500 MHz, DMSO-d₆) δ ppm 1.14 (t, 3H), 1.94 (s, 3H), 3.84 (m, 2H), 6.72 (br. s., 2H), 7.22 (m, 1 H), 7.29 (m, 2H), 7.40 (m, 1H), 7.44 (m, 1H), 7.56 (m, 1H), 7.67 (d, 1H), 8.30 (d, 1H); MS lc) (ES+) m/z 441, 443 [M+H]⁺.

### Example 21i

### 5-(3-Amino-1-(3-bromo-4-fluorophenyl)-4-fluoro-1H-isoindol-1-yl)-1,3-dimethylpyridin-2(1H)-one

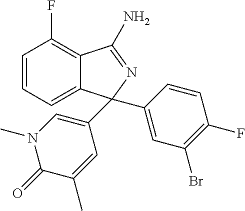

5-Bromo-1,3-dimethylpyridin-2(1H)-one (1.069 g, 5.29 mmol, C. Morrow et al. J. Org. Chem., 1974, 39 (14), pp 2116-2118) was stirred under Ar (g) for 3 min and dissolved in THF (8 mL). Isopropylmagnesiumchloride lithiumchloride complex (1.3 M in THF) (3.74 mL, 4.87 mmol) was added dropwise and the mixture were stirred for 30 sec. N-((3-bromo-4-fluorophenyl)(2-cyano-3-fluorophenyl)methylene)-2-methylpropane-2-sulfinamide (0.9 g, 2.12 mmol, WO 2010056196) in THF (4 mL) was added. The mixture was stirred overnight, HCl in MeOH (1.25 M, 8.46 mL, 10.58 mmol) was added and the mixture was stirred for 1 h. The reaction was quenched with NaHCO₃ (aq.sat.) and extracted with EtOAc. The organic phases were pooled, dried over MgSO₄, filtered and concentrated. Chromatography on a silica gel column eluted with a gradient of 0-3% (0.1 M NH₃ in MeOH) in DCM gave the title compound (664 mg, 71% yield). ¹H NMR (400 MHz, DMSO-d₆) δ ppm 1.93 (s, 3H), 3.35 (s, 3H), 6.61 (br. s., 2H), 7.21-7.32 (m, 4H), 7.35-7.40 (m, 1H), 7.50-7.56 (m, 2H), 7.58-7.62 (m, 1H);); MS (ES+) m/z 444, 446 [M+H]⁺.

### Example 22i

### 5-Bromo-3-(difluoromethyl)-2-methoxypyridine

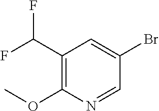

To 5-bromo-2-methoxynicotinaldehyde (5 g, 23 mmol) in dry CH₂Cl₂ (100 mL) at 0° C. under argon was diethylaminosulphur trifluoride (3.69 mL, 30.1 mmol) added over 1 min. The reaction mixture was stirred for three days while the reaction warmed to r.t. The reaction was quenched by the addition of sat. aqueous sodium bicarbonate solution. The reaction mixture was combined with another reaction based on 5-bromo-2-methoxynicotinaldehyde (100 mg, 0.46 mmol) prior to workup. The phases were separated and the water phase was further extracted with CH₂Cl₂ (x 3). The organic layers were pooled, dried (Na₂SO₄), filtered and concentrated to give the title compound (5.71 g, quant. yield): ¹H NMR (400 MHz, DMSO-d₆) δ ppm 3.94 (s, 3H), 7.04 (t, 1 H), 8.10-8.16 (m, 1H), 8.48 (m, 1H); MS (ES+) m/z 238 [M+H]⁺.

### Example 23i

### 5-Bromo-3-(difluoromethyl)pyridin-2(1H)-one

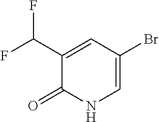

Hydrobromic acid (33% in glacial acetic acid) (63.4 mL, 361 mmol) was added to 5-bromo-3-(difluoromethyl)-2-methoxypyridine (6.37 g, 26.8 mmol, Example 22i). The resulting reaction mixture was stirred at r.t. for 5 h, then at 40° C. for 75 min, then at 50° C. for 25 min. The reaction was allowed to cool, then the reaction mixture was concentrated in vacuo. The residue was partitioned between NaHCO₃ (aq. sat.) and CHCl₃. The aqueous phase was extracted twice with CHCl₃, the combined organics were passed through a phase separator and concentrated to give the title compound (5.68 g, 95% yield). ¹H NMR (500 MHz, DMSO-d₆) δ ppm 6.81 (t, 1H), 7.78-7.83 (m, 1H), 7.89 (m, 1H), 12.46 (br. s., 1H); MS (ES+) m/z 224 [M+H]⁺.

### Example 24i

### 5-Bromo-2-(tert-butyldimethylsilyloxy)-3-(difluoromethyl)pyridine

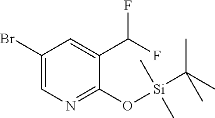

The title compound was synthesized as described for Example 81 in 79% yield starting from 5-bromo-3-(difluoromethyl)pyridin-2(1H)-one (1.03 g, 4.62 mmol, Example 23i,) and tert-butyldimethylchlorosilane (0.835 g, 5.54 mmol), with the exception that the product was co-evaporated with toluene twice: ¹H NMR (600 MHz, DMSO-d₆) δ ppm 0.31 (s, 6H), 0.96 (s, 9 H), 6.97 (t, 1H), 8.12 (s, 1H), 8.41 (s, 1H); MS (ES+) m/z 338 [M+H]⁺.

### Example 25i

### 5-(3-Amino-1-(3-bromophenyl)-4-fluoro-1H-isoindol-1-yl)-3-(difluoromethyl)pyridin-2(1H)-one

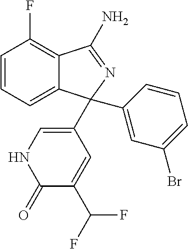

n-Butyllithium (2.5 M in hexanes, 1.38 mL, 3.45 mmol) was added dropwise over 3 min to a solution of 5-bromo-2-(tert-butyldimethylsilyloxy)-3-(difluoromethyl)pyridine (1 g, 2.96 mmol, Example 24i) in dry THF (10 mL) at −66° C. under Ar (g). After 10 min. a solution of N-((3-bromophenyl)(2-cyano-3-fluorophenyl)methylene)-2-methylpropane-2-sulfinamide (1.00 g, 2.46 mmol, WO2010/056196) in dry THF (5 mL) was added dropwise over 3 min. After 1 h at −66° C. the flask was taken up from the cooling bath and left to stir at rt. After 2.5 h, hydrogen chloride in methanol solution (1.25 M, 5.91 mL, 7.39 mmol) was added and the reaction was stirred overnight. More hydrogen chloride in methanol solution (1.25 M, 4 mL, 5.00 mmol) was added. 4.5 h later water was added and the pH was adjusted to approximately 8 with NaOH (10%, aq.), brine was added, and the mixture was extracted with EtOAc (×3), dried (Na₂SO₄), filtered and concentrated. The crude material was combined with a similar reaction based on N-((3-bromophenyl)(2-cyano-3-fluorophenyl)methylene)-2-methylpropane-2-sulfinamide (200 mg, 0.49 mmol). Flash silica gel chromatography using CHCl₃/MeOH 9:1 as eluent gave the title compound (0.913 g, 69% yield): MS (ES+) m/z 448 [M+H]⁺.

### Example 26i

### 5-(3-Amino-1-(3-bromophenyl)-4-fluoro-1H-isoindol-1-yl)-3-(difluoromethyl)-1-ethylpyridin-2(1H)-one

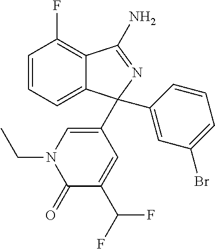

Iodoethane (0.020 mL, 0.25 mmol) was added to a 0° C. mixture of sodium hydride (10.7 mg, 0.27 mmol) and 5-(3-amino-1-(3-bromophenyl)-4-fluoro-1H-isoindol-1-yl)-3-(difluoromethyl)pyridin-2(1H)-one (100 mg, 0.22 mmol, Example 25i) in dry DMF (2.7 mL) under Ar (g). The reaction mixture was left at 4° C. overnight and then the reaction was left to warm to r.t. After 7 h the reaction mixture was combined with a reaction based on a mixture of 5-(3-amino-1-(3-bromophenyl)-4-fluoro-1H-isoindol-1-yl)-3-(difluoromethyl)pyridin-2(1H)-one (100 mg, 0.22 mmol, Example 25i), potassium carbonate (62 mg, 0.45 mmol) in DME (0.5 mL) and iodoethane (0.020 mL, 0.25 mmol), that had been stirred at room temperature overnight. The combined reactions were quenched with water and the pH was adjusted to approx. 7 using HCl (1 M) and NaHCO₃ (aq sat). The mixture was extracted (×3) with CHCl₃. The organics were dried (Na₂SO₄), filtered, concentrated and co-evaporated once with toluene. Purification by flash silica gel chromatography using a gradient of CHCl₃/MeOH 50:1-40:1-30:1-20:1-10:1 gave the title compound (99 mg, 48% yield): ¹H NMR (500 MHz, DMSO-d₆) δ ppm 1.16 (t, 3H), 3.91 is (m, 2H), 6.71 (br. s., 2H), 6.83 (t, 1H), 7.25-7.32 (m, 2H), 7.38 (d, 1H), 7.43-7.48 (m, 2H), 7.52-7.65 (m, 4H); MS (ES+) m/z 476 [M+H]⁺.

### Example 27i

### 3-Bromo-5-(prop-1-ynyl)pyridine

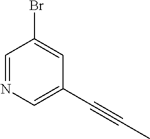

3,5-Dibromopyridine (30 g, 127 mmol), copper(I) iodide (7.24 g, 38.0 mmol) and tetrakis-(triphenylphosphine)palladium(0) (4.39 g, 3.80 mmol) were mixed in toluene (120 mL) under nitrogen atmosphere. 1-(Trimethylsilyl)-1-propyne (26.36 mL, 164.5 mmol), triethylamine (53.0 mL, 380 mmol) and tetra-n-butylammonium fluoride (12.66 mL, 12.66 mmol) were added. The mixture was heated to reflux and stirred under nitrogen overnight. Water (100 mL) was added to the reaction mixture was filtered and the phases separated. The organic phase was washed with 1 M HCl aq. (100 mL). The organic phase was concentrated and dissolved in methanol (200 mL), filtered and concentrated. The mixture was dissolved in DCM and evaporated with silica gel to dryness, and then transferred to a silica gel column (300 g). The product was eluted with a gradient of 0-5% EtOAc in heptane. The fractions containing the pure product was combined and evaporated to give the title compound (16.39 g, 66% yield): ¹H NMR (500 MHz, CDCl₃) δ ppm 2.08 (s, 4H), 7.82 (t, 1H), 8.52 (d, 1H), 8.55 (d, 1H); MS (APCI+) m/z 197.0 [M+H]⁺.

### Example 28i

### 5-(Prop-1-ynyl)pyridin-3-yl boronic acid

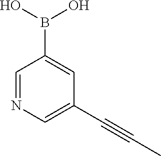

3-Bromo-5-(prop-1-ynyl)pyridine (25 g, 117 mmol, Example 27i), 2-methyl-tetrahydrofuran (60 mL), toluene (200 mL) and triisopropyl borate (33.2 mL, 140 mmol) were mixed. The mixture was cooled to −50° C. To the cold mixture was added n-BuLi (59.8 mL, 149.5 mmol) dropwise during 30 minutes. The mixture was stirred for 60 minutes at −50° C. 2M HCl aq. (100 mL) was added. The mixture was then allowed to reach r.t. and stirred for 20 minutes. The organic and water phase were separated. The organic phase was extracted with NaOH (2M aq.) (2×100 mL). The water phases were combined and pH adjusted to pH 5. The product was extracted with 2-methyl THF (2×100 mL). The organic phase was dried with sodium sulphate, filtered and concentrated to give the title compound (16.47 g, 87% yield): ¹H NMR (500 MHz, CD₃OD) δ ppm 2.11 (s, 3H), 8.21 (br. s., 1H), 8.53 (m, 2H); MS (APCI+) m/z 162.2 [M+H]⁺.

### Example 29i

### 2-Bromo-6-(prop-1-ynyl)pyridine

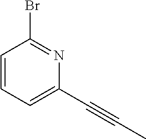

To a mixture of 2-bromo-6-iodopyridine (3 g, 10.5 mmol), copper (I) iodide (604 mg, 3.17 mmol), tetrakis(triphenylphosphine)palladium(0) (611 mg, 0.53 mmol) in toluene (22.5 mL), under Ar (g), was 1-(trimethylsilyl)-1-propyne (1.57 mL, 10.6 mmol), triethylamine (4.85 mL, 34.9 mmol) and tetrabutylammonium fluoride (10.6 mL, 10.6 mmol) added and the mixture was stirred at r.t. for 4 days. The reaction was combined with a similar reaction based on 2-bromo-6-iodopyridine (0.2 g, 0.70 mmol) prior to workup. Water, Et₂O and CHCl₃ were added and the phases were separated. The organic phase was dried (Na₂SO₄), filtered and concentrated. Purification twice by flash silica gel chromatography using a gradient of heptane/CH₂Cl₂ 1:1-2:3 gave the title compound (1.60 g, 72% yield): ¹H NMR (500 MHz, DMSO-d₆) δ ppm 2.09 (s, 3 H), 7.49 (d, 1H), 7.61 (d, 1H), 7.73 (t, 1H); MS (ES+) m/z 195.9 [M+H]⁺.

### Example 30i

### 2-Chloro-4-(prop-1-ynyl)pyridine

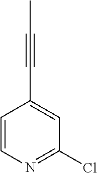

4-Bromo-2-chloropyridine (1.00 g, 5.2 mmol), 1-(trimethylsilyl)-1-propyne (0.85 mL, 5.7 mmol), copper(I) iodide (99 mg, 0.52 mmol), Pd(Ph₃P)₄ (90 mg, 0.08 mmol) were taken up in toluene (14 mL). Tetrabutylammonium fluoride (1 M in THF, 6.0 mL, 6.0 mmol) was added and the reaction mixture heated in a microwave reactor at 100° C. for 20 min. After cooling, the reaction mixture was filtered and evaporated. Purification by column chromatography using a gradient of EtOAc in heptane (0-50%) gave the title compound (530 mg, 67% yield): ¹H NMR (500 MHz, DMSO-d₆) δ ppm 2.11 (s, 3H), 7.38 (dd, 1H), 7.51 (s, 1H), 8.37 (d, 1H); MS (ES+) m/z 152 [M+H]⁺.

### Example 1(R)-5-(3-Amino-4-fluoro-1-(3-(5-(prop-1-ynyl)pyridin-3-yl)phenyl)-1H-isoindol-1-yl)-1,3-dimethylpyridin-2(1H)-one

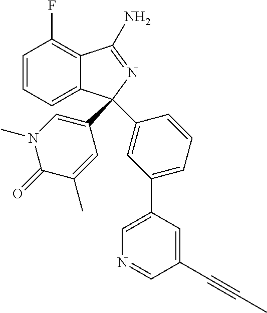

A mixture of (R)-5-(3-amino-1-(3-bromophenyl)-4-fluoro-1H-isoindol-1-yl)-1,3-dimethyl-pyridin-2(1H)-one (150 mg, 0.35 mmol, Example 19i), 5-(prop-1-ynyl)pyridin-3-ylboronic acid (113 mg, 0.70 mmol, Example 28i), dichloro[1,1′-bis(diphenylphosphino)ferrocene]palladium (II) dichloromethane adduct (29 mg, 0.04 mmol) and K₂CO₃, 2 M in water (0.53 mL, 1.06 mmol) in dioxane (5 mL) was heated in a microwave reactor at 130° C. for 15 min. Purification by preparative HPLC gave the title compound as an unknown mixture with the other enantiomer (22 mg, 14% yield). ¹H NMR (500 MHz, DMSO-d₆) δ ppm 1.93 (s, 3H), 2.11 (s, 3H), 3.36 (s, 3 H), 6.53 (br. s, 2H), 7.15-7.34 (m, 3H), 7.36-7.48 (m, 2H), 7.52 (m, 1H), 7.56-7.65 (m, 2 H), 7.71 (d, 1H), 7.96 (m, 1H), 8.56 (m, 1H), 8.71 (m, 1H); MS (ES+) m/z 463 [M+H]⁺.

### Example 2

### 5-(3-Amino-4-fluoro-1-(5′-(prop-1-ynyl)-2,3′-bipyridin-4-yl)-1H-isoindol-1-yl)-1-ethyl-3-methylpyridin-2(1H)-one

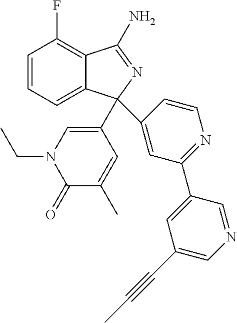

### Method A

5-(3-Amino-1-(2-bromopyridin-4-yl)-4-fluoro-1H-isoindol-1-yl)-1-ethyl-3-methylpyridin-2(1H)-one (98 mg, 0.22 mmol, Example 20i), 5-(prop-1-ynyl)pyridin-3-ylboronic acid (64 mg, 0.40 mmol, Example 28i), [1,1′-bis(diphenylphosphino)ferrocene]palladium(II) chloride (9.13 mg, 0.01 mmol) and cesium carbonate (145 mg, 0.44 mmol) were dissolved in DME:EtOH:water (6:3:1) (2 mL) and heated to 130° C. for 20 min in a micro wave reactor. Purification by preparative HPLC gave the title compound (34 mg, 32% yield). ¹H NMR (500 MHz, DMSO-d₆) δ ppm 1.15 (t, 3H), 1.93 (s, 3H), 2.11 (s, 3H), 3.85 (m, 2H), 6.68 (br. s., 2H), 7.23 (m, 1H), 7.29 (m, 2H), 7.42 (m, 1H), 7.56 (m, 1H), 7.81 (d, 1H), 7.91 (m, 1H), 8.30 (t, 1H), 8.62 (m, 2 H), 9.08 (d, 1H); MS (ES+) m/z 478 [M+H]⁺.

### Method B

5-(3-Amino-1-(2-bromopyridin-4-yl)-4-fluoro-1H-isoindol-1-yl)-1-ethyl-3-methylpyridin-2(1H)-one (317 mg, 0.72 mmol, Example 20i), 5-(prop-1-ynyl)pyridin-3-yl boronic acid (127 mg, 0.79 mmol, Example 28i), 2-dicyclohexylphosphino-2′,6′-dimethoxybiphenyl (S-phos) (30 mg, 0.07 mmol), K₂CO₃ (199 mg, 1.44 mmol) and Pd(OAc)₂ (8.1 mg, 0.04 mmol) in dioxane (10 mL) and water (2 mL) were heated in a microwave reactor at 100° C. for 15 min. The reaction mixture was separated between EtOAc and water, the organic layer was washed with brine, dried over Na₂SO₄, filtered and concentrated. Purification by silica gel column chromatography using a gradient of 0-60% (DCM containing 10% 0.7 M NH₃ in MeOH) in DCM gave the title compound (233 mg, 68% yield): ¹H NMR (500 MHz, DMSO-d₆) δ ppm 1.15 (t, 3H), 1.93 (s, 2 H), 2.11 (s, 2H), 3.86 (m, 2H), 6.68 (br. s., 1H), 7.23 (d, 1H), 7.26-7.32 (m, 2H), 7.42 (m, 1 H), 7.56 (m, 1H), 7.81 (d, 1H), 7.91 (s, 1H), 8.30 (t, 1H), 8.58-8.66 (m, 2H), 9.08 (d, 1H). MS (ES+) m/z 478 [M+H]⁺.

### Example 3

### (R)-5-(3-Amino-4-fluoro-1-(3-(5-(prop-1-ynyl)pyridin-3-yl)phenyl)-1H-isoindol-1-yl)-1-ethyl-3-methylpyridin-2(1H)-one

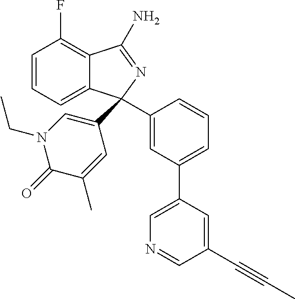

### Method A

A stirred mixture of (R)-5-(3-amino-1-(3-bromophenyl)-4-fluoro-1H-isoindol-1-yl)-1-ethyl-3-methylpyridin-2(1H)-one (5 g, 11.36 mmol, Example 21), 5-(prop-1-ynyl)pyridin-3-ylboronic acid (2.64 g, 14.8 mmol, Example 28i), [1,1′bis(diphenylphosphino)ferrocene]palladium(II) chloride (0.467 g, 0.57 mmol) in 2-methyl-tetrahydrofuran (50 mL) and potassium carbonate (2 M, 17.03 mL, 34.07 mmol) was degassed under vacuum, flushed with nitrogen and heated to reflux for 120 min. The phases were separated. The organic phase was extracted with citric acid 2 M (30 mL). n-Heptane (40 mL) and citric acid 2 M (30 mL) were added to the organic phase, the phases were separated. The citric acid phases were combined and the pH was adjusted to approximately 9 using 4 M NaOH. The water mixture was extracted with ethyl acetate (2×40 mL) and dichloromethane (40 mL). The organic phases were combined, dried (Na₂SO₄), concentrated and filtered through a plug of silica gel with dichloromethane as eluent. The crude was purified by silica gel chromatography using a gradient of DCM to DCM:MeOH:7 M NH₃ in MeOH (1000:40:10). The fractions were combined, and concentrated, EtOAc (70 mL) and water (70 mL) were added to the crude material and the phases were separated. The organic phase was concentrated, co-evaporated twice with methanol (10 mL) and dried under vacuum to give the title compound (3.42 g, 63% yield). ¹H NMR (400 MHz, DMSO-d₆) δ ppm 1.14 (t, 3H), 1.93 (s, 3H), 2.10 (s, 3H), 3.84 (m, 2H), 6.59 (br. s., 2H), 7.20-7.30 (m, 3H), 7.38-7.46 (m, 2H), 7.52 (m, 1H), 7.56-7.63 (m, 2H), 7.72 (d, 1H), 7.95 (t, 1H), 8.56 (d, 1H), 8.70 (d, 1H); MS (ES+) m/z 477 [M+H]⁺.

### Method B

A stirred mixture of (R)-5-(3-amino-1-(3-bromophenyl)-4-fluoro-1H-isoindol-1-yl)-1-ethyl-3-methylpyridin-2(1H)-one (20 g, 45.4 mmol, Example 21), 5-(prop-1-ynyl)pyridin-3-ylboronic acid (8.77 g, 54.5 mmol, Example 28i), sodium tetrachloropalladate(II) (0.668 g, 2.27 mmol), 3-(di-tert-butylphosphonium)propane sulfonate (1.219 g, 4.54 mmol), in 2-methyl-tetrahydrofuran (400 mL) and 2 M aqueous potassium carbonate (68.1 mL, 136 mmol) was degassed under vacuum, flushed with nitrogen and heated to reflux for 120 min. Water (100 mL) and toluene (100 mL) were added. The phases were separated. The organic phase was dried (Na₂SO₄), and concentrated. The crude product was purified by silica gel chromatography using a gradient of DCM to DCM:MeOH:(7 M NH₃ in MeOH) (1000:40:10). The fractions were combined, and concentrated, co-evaporated twice with methanol (30 mL) and dried in vacuo to give the title compound (20.2 g, 93% yield): ¹H NMR (400 MHz, DMSO-d₆) δ ppm 1.14 (t, 3H), 1.93 (s, 3 H), 2.10 (s, 3H), 3.84 (m, 2H), 6.59 (br. s., 2H), 7.20-7.30 (m, 3H), 7.38-7.46 (m, 2H), 7.52 (m, 1H), 7.56-7.63 (m, 2H), 7.72 (d, 1H), 7.95 (t, 1H), 8.56 (d, 1H), 8.70 (d, 1H); MS (ES+) m/z 477 [M+H]⁺.

### Example 4

### (R)-5-(3-Amino-4-fluoro-1-(3-(4-fluoro-5-methylpyridin-3-yl)phenyl)-1H-isoindol-1-yl)-1-ethyl-3-methylpyridin-2(1H)-one

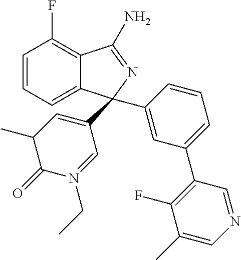

(R)-5-(3-Amino-1-(3-bromophenyl)-4-fluoro-1H-isoindol-1-yl)-1-ethyl-3-methylpyridin-2(1H)-one (122 mg, 0.28 mmol, 75% enantiomeric purity, Example 21), 4-fluoro-3-methyl-5-(tributyl-stannyl)pyridine (116 mg, 0.29 mmol, Example 31), tetrakis(triphenylphosphine)palladium(0) (32 mg, 0.03 mmol) and DMF (4 mL) were put in microwave vial and irradiated in a microwave reactor at 150° C. for 20 min. Purification by preparative HPLC gave the title compound as a mixture with the other enantiomer (4 mg, 3% yield): ¹H NMR (500 MHz, DMSO-d₆) δ ppm 1.14 (t, 3H), 1.94 (s, 3H), 2.27 (s, 3H), 3.84 (q, 2H), 6.56 (br. s., 2H), 7.22-7.29 (m, 3H), 7.39-7.46 (m, 3H), 7.48-7.55 (m, 2H), 7.63 (d, 1H), 8.46 (t, 2H); MS (ES+) m/z 471 [M+H]⁺.

### Example 5

### 5-(3-(3-Amino-4-fluoro-1-(1-isopropyl-5-methyl-6-oxo-1,6-dihydropyridin-3-yl)-1H-isoindol-1-yl)phenyl)nicotinonitrile

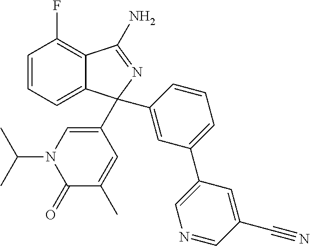

5-Cyanopyridin-3-ylboronic acid (33 mg, 0.22 mmol), 5-(3-amino-1-(3-bromophenyl)-4-fluoro-1H-isoindol-1-yl)-1-isopropyl-3-methylpyridin-2(1H)-one (77 mg, 0.17 mmol, Example 4i), dichloro[1,1′-bis(diphenylphosphino)ferrocene]palladium (II) dichloromethane adduct (6.9 mg, 8.47 μmol) and cesium carbonate (166 mg, 0.51 mmol) were mixed in DME:EtOH:water (6:3:1) (2.5 mL) and heated in a microwave reactor at 150° C. for 15 minutes. Purification twice by preparative HPLC gave the title compound (19 mg, 23% yield). ¹H NMR (500 MHz, DMSO-d₆) δ ppm 1.14 (d, 3H), 1.21 (d, 3H), 1.92 (s, 3H), 4.97 (m, 1H), 6.58 (br. s., 2H), 7.21-7.29 (m, 3H), 7.43-7.50 (m, 2H), 7.53 (td, 1H), 7.62-7.68 (m, 2H), 7.75 (d, 1H), 8.53 (t, 1H), 8.99 (d, 1H), 9.05 (d, 1H); MS (ES+) m/z 478 [M+H]⁺.

### Example 6

### 5-(3-Amino-4-fluoro-1-(3-(pyrimidin-5-yl)phenyl)-1H-isoindol-1-yl)-1-ethyl-3-methylpyridin-2(1H)-one

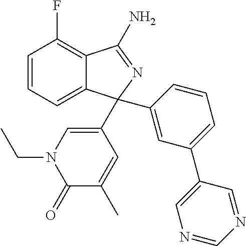

Pyrimidin-5-ylboronic acid (19 mg, 0.15 mmol), dichloro[1,1′-bis(diphenylphosphino)-ferrocene]palladium (II) dichloromethane adduct (5 mg, 5.8 μmol) and cesium carbonate (113 mg, 0.35 mmol) and 5-(3-amino-1-(3-bromophenyl)-4-fluoro-1H-isoindol-1-yl)-1-ethyl-3-methylpyridin-2(1H)-one (51.0 mg, 0.12 mmol, Example 15i) in DME:EtOH:water (6:3:1) (2.5 mL) were microwaved at 150° C. for 20 min. The mixture was diluted with EtOAc (10 mL) and washed with brine (5 mL). The aqueous layer was extracted with EtOAc (10 mL), The combined organics were concentrated. The crude was purified by flash chromatography on silica gel using a gradient from DCM to 100% (0.1 N NH₃ in 10% MeOH in DCM) to give the title compound is (36 mg, 71% yield). ¹H NMR (500 MHz, DMSO-d₆) δ ppm 1.13 (t, 3H), 1.92 (s, 3H), 3.83 (m, 2H), 6.55 (br. s., 2H), 7.21-7.29 (m, 3H), 7.42-7.48 (m, 2H), 7.51 (td, 1H), 7.62-7.67 (m, 2H), 7.73 (d, 1H), 9.02 (s, 2H), 9.17 (s, 1H); MS (ES+) m/z 440 [M+H]⁺.

### Example 7

### 5-(3-Amino-4-fluoro-1-(3-(5-fluoropyridin-3-yl)phenyl)-1H-isoindol-1-yl)-1-isopropyl-3-methylpyridin-2(1H)-one

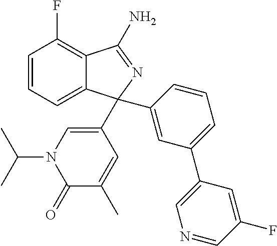

The title compound was synthesized as described for Example 6 in 63% yield starting from 5-fluoropyridin-3-ylboronic acid (29 mg, 0.20 mmol) and 5-(3-amino-1-(3-bromophenyl)-4-fluoro-1H-isoindol-1-yl)-1-isopropyl-3-methylpyridin-2(1H)-one (77 mg, 0.17 mmol, Example 41), with the exception that the crude product was purified by preparative HPLC. ¹H NMR (500 MHz, DMSO-d₆) δ ppm 1.14 (d, 3H), 1.21 (d, 3H), 1.93 (s, 3H), 4.97 (m, 1H), 6.58 (br. s., 2H), 7.21-7.30 (m, 3H), 7.40-7.48 (m, 2H), 7.53 (td, 1H), 7.59 (m, 1H), 7.62 (ddd, 1H), 7.73 (d, 1H), 7.94 (m, 1H), 8.56 (d, 1H), 8.65 (t, 1H); (ES+) m/z 471 [M+H]⁺.

### Example 8

### 5-(3-(3-Amino-1-(1-ethyl-5-methyl-6-oxo-1,6-dihydropyridin-3-yl)-4-fluoro-1H-isoindol-1-yl)phenyl)nicotinonitrile

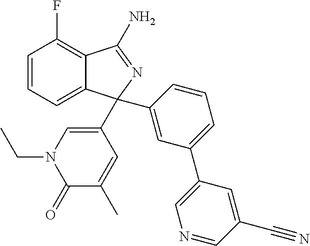

The title compound was synthesized as described for Example 6 in 11% yield starting from 5-cyanopyridin-3-ylboronic acid (17 mg, 0.12 mmol) and 5-(3-amino-1-(3-bromophenyl)-4-fluoro-1H-isoindol-1-yl)-1-ethyl-3-methylpyridin-2(1H)-one (51 mg, 0.12 mmol, Example 15i), with the exception that the crude product was purified by flash chromatography on silica gel followed by preparative HPLC. ¹H NMR (500 MHz, DMSO-d₆) δ ppm 1.13 (m, 3H), 1.91 (m, 3H), 3.83 (m, 2H), 6.55 (br. s., 2H), 7.19-7.28 (m, 4H), 7.42-7.55 (m, 3H), 7.63-7.67 (m, 2H), 7.74 (d, 1H), 8.54 (m, 1H), 8.99 (d, 1H), 9.05 (d, 1H); MS (ES+) m/z 464 [M+H]⁺.

### Example 9

### 5-(3-Amino-4-fluoro-1-(3-(5-fluoropyridin-3-yl)phenyl)-1H-isoindol-1-yl)-1-ethyl-3-methylpyridin-2(1H)-one

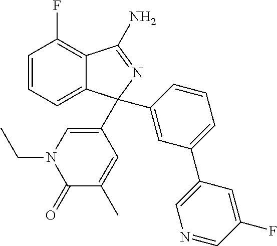

The title compound was synthesized as described for Example 6 in 70% yield starting from 5-fluoropyridin-3-ylboronic acid (16 mg, 0.12 mmol) and 5-(3-amino-1-(3-bromophenyl)-4-fluoro-1H-isoindol-1-yl)-1-ethyl-3-methylpyridin-2(1H)-one (51 mg, 0.12 mmol, Example 15i), with the exception that the crude product was purified twice by flash chromatography on silica gel using a gradient from DCM to 100% (0.1 N NH₃ in 10% MeOH in DCM). ¹H NMR (500 MHz, DMSO-d₆) δ ppm 1.13 (m, 3H), 1.92 (s, 3H), 3.83 (m, 2H), 6.55 (br. s., 2H), 7.21-7.28 (m, 3 H), 7.40-7.46 (m, 2H), 7.51 (m, 1H), 7.59-7.64 (m, 2H), 7.72 (d, 1H), 7.94 (ddd, 1H), 8.56 (d, 1H), 8.65 (m, 1H); MS (ES+) m/z 457 [M+H]⁺.

### Example 10

### 5-(3-(3-Amino-4-fluoro-1-(1-methyl-6-oxo-5-(trifluoromethyl)-1,6-dihydropyridin-3-yl)-1H-isoindol-1-yl)phenyl)nicotinonitrile

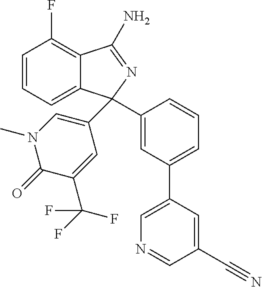

tert-Butyl 1-(3-bromophenyl)-4-fluoro-1-(1-methyl-6-oxo-5-(trifluoromethyl)-1,6-dihydro-pyridin-3-yl)-1H-isoindol-3-ylcarbamate (348 mg, 0.6 mmol, Example 5i), 5-cyanopyridin-3-ylboronic acid (107 mg, 0.72 mmol), [1,1′-bis(diphenylphosphino)ferrocene]palladium(II) chloride (37 mg, 0.05 mmol), potassium carbonate (249 mg, 1.80 mmol), DME (1.5 mL), water (0.75 mL) and EtOH (0.25 mL) were heated in a microwave reactor at 150° C. for 15 min. The mixture was diluted with MeOH (2 mL), filtered and purified by preparative HPLC to give the title compound (68 mg, 23% yield). ¹H NMR (400 MHz, DMSO-d₆) δ ppm 3.46 (s, 3H), 6.70 (br. s., 2H), 7.25-7.32 (m, 1H), 7.44-7.59 (m, 3H), 7.69 (dt, 1H), 7.72-7.80 (m, 4H), 8.57 (t, 1H), 9.01 (d, 1H), 9.10 (d, 1H); MS (ES+) m/z 504 [M+H]⁺.

### Example 11

### 5-(3-Amino-4-fluoro-1-(3-(5-methoxypyridin-3-yl)phenyl)-1H-isoindol-1-yl)-1-methyl-3-(trifluoromethyl)pyridin-2(1H)-one

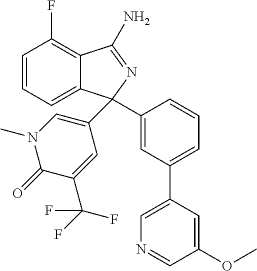

The title compound was synthesized as described for Example 10 in 19% yield starting from tert-butyl 1-(3-bromophenyl)-4-fluoro-1-(1-methyl-6-oxo-5-(trifluoromethyl)-1,6-dihydropyridin-3-yl)-1H-isoindol-3-ylcarbamate (232 mg, 0.4 mmol, Example 5i) and 3-methoxypyridin-5-boronic acid (73 mg, 0.48 mmol). ¹H NMR (400 MHz, DMSO-d₆) δ ppm 3.45 (s, 3H), 3.88 (s, 3 H), 6.70 (br. s., 2H), 7.28 (dd, 1H), 7.40-7.46 (m, 2H), 7.50 (dd, 1H), 7.55 (td, 1H), 7.59-7.65 (m, 2H), 7.71 (d, 1H), 7.78 (dd, 2H), 8.29 (d, 1H), 8.38 (d, 1H); MS (ES+) m/z 509 [M+H]⁺.

### Example 12

### 5-(3-Amino-4-fluoro-1-(3-(5-fluoropyridin-3-yl)phenyl)-1H-isoindol-1-yl)-1-methyl-3-(trifluoromethyl)pyridin-2(1H)-one

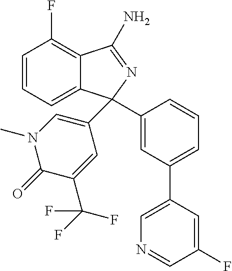

The title compound was synthesized as described for Example 10 in 32% yield starting from tert-butyl 1-(3-bromophenyl)-4-fluoro-1-(1-methyl-6-oxo-5-(trifluoromethyl)-1,6-dihydropyridin-3-yl)-1H-isoindol-3-ylcarbamate (232 mg, 0.4 mmol, Example 5i) and 5-fluoropyridine-3-boronic acid (68 mg, 0.48 mmol). ¹H NMR (500 MHz, DMSO-d₆) δ ppm 3.46 (s, 3H), 6.67 (br. s., 2H), 7.28 (dd, 1H), 7.42-7.50 (m, 2H), 7.55 (td, 1H), 7.65 (dt, 1H), 7.70 (s, 1H), 7.72-7.76 (m, 2 H), 7.78 (d, 1H), 7.98 (dt, 1H), 8.57 (d, 1H), 8.70 (t, 1H); MS (ES+) m/z 497 [M+H]⁺.

### Example 13

### 5-(3-Amino-4-fluoro-1-(3-(pyrimidin-5-yl)phenyl)-1H-isoindol-1-yl)-1-methyl-3-(trifluoromethyl)pyridin-2(1H)-one

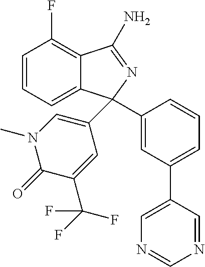

The title compound was synthesized as described for Example 10 in 34% yield starting from tert-butyl 1-(3-bromophenyl)-4-fluoro-1-(1-methyl-6-oxo-5-(trifluoromethyl)-1,6-dihydropyridin-3-yl)-1H-isoindol-3-ylcarbamate (232 mg, 0.4 mmol, Example 5i) and pyrimidine-5-boronic acid (64 mg, 0.52 mmol). ¹H NMR (600 MHz, DMSO-d₆) δ ppm 3.46 (s, 3H), 6.67 (br. s., 2H), 7.28 (t, 1H), 7.44-7.51 (m, 2H), 7.55 (td, 1H), 7.68 (dt, 1H), 7.72-7.77 (m, 3H), 7.79 (d, 1H), 9.06 (s, 2H), 9.18 (s, 1H); MS (ES+) m/z 480 [M+H]⁺.

### Example 14

### 5-(3-Amino-4-fluoro-1-(3-(pyrimidin-5-yl)phenyl)-1H-isoindol-1-yl)-1,3-dimethylpyridin-2(1H)-one

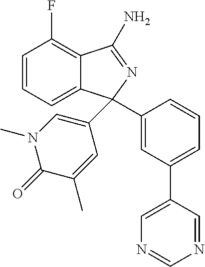

The title compound was synthesized as described for Example 10 in 35% yield starting from 5-(3-amino-1-(3-bromophenyl)-4-fluoro-1H-isoindol-1-yl)-1,3-dimethylpyridin-2(1H)-one (110 mg, 0.26 mmol, Example 9i method A) and pyrimidine-5-boronic acid (38 mg, 0.31 mmol) with is the exception that it was heated in a microwave reactor at 120° C. for two times 15 min. ¹H NMR (400 MHz, DMSO-d₆) δ ppm 1.92 (s, 3H), 3.36 (s, 3H), 6.57 (br. s., 2H), 7.21-7.30 (m, 3H), 7.42-7.56 (m, 3H), 7.62-7.69 (m, 2H), 7.72 (d, 1H), 9.04 (s, 2H), 9.18 (s, 1H); MS (ES+) m/z 426 [M+H]⁺.

### Example 15

### 5-(3-Amino-1-(5′-chloro-2,3′-bipyridin-4-yl)-4-fluoro-1H-isoindol-1-yl)-1,3-dimethylpyridin-2(1H)-one

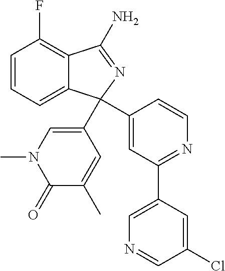

5-(3-Amino-1-(2-bromopyridin-4-yl)-4-fluoro-1H-isoindol-1-yl)-1,3-dimethylpyridin-2(1H)-one (190 mg, 0.44 mmol, Example 10i), 5-chloropyridin-3-ylboronic acid (77 mg, 0.49 mmol) and (1,1′-bis(diphenylphosphino)ferrocene)-dichloropalladium(II) (37 mg, 0.04 mmol) were mixed in THF (3 mL). Sodium carbonate (aq., 2 M) (0.667 mL, 1.33 mmol) was added and the mixture was run in a microwave reactor for 40 min at 140° C. The resulting mixture was filtered through diatomaceous earth, concentrated and purified by preparative HPLC to give the title compound (14 mg, 7% yield). ¹H NMR (600 MHz, DMSO-d₆) δ ppm 1.93 (s, 3H), 3.37 (s, 3H), 6.66 (br. s., 2H), 7.23 (m, 1H), 7.26-7.33 (m, 2H), 7.44 (dd, 1H), 7.56 (td, 1H), 7.79 (d, 1H), 7.97 (m, 1H), 8.44 (t, 2H), 8.62 (m, 1H), 8.70 (dd, 1H), 8.99 (d, 1H), 9.13 (d, 1H); MS (ES+) m/z 460 [M+H]⁺.

### Example 16

### 5-(3-Amino-4-fluoro-1-(5′-(prop-1-ynyl)-2,3′-bipyridin-4-yl)-1H-isoindol-1-yl)-1,3-diethylpyridin-2(1H)-one

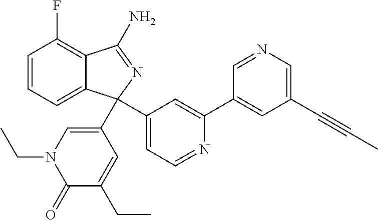

5-(3-Amino-1-(2-bromopyridin-4-yl)-4-fluoro-1H-isoindol-1-yl)-1,3-diethylpyridin-2(1H)-one (70 mg, 0.15 mmol, Example 11i), 5-(prop-1-ynyl)pyridin-3-ylboronic acid (50 mg, 0.31 mmol, Example 28i), [1,1′-bis(diphenylphosphino)ferrocene]palladium(II) chloride (6 mg, 7.69 μmol), cesium carbonate (150 mg, 0.46 mmol) and DME:EtOH:water 6:3:1 (5 mL) were heated in a microwave reactor at 150° C. for 30 min. EtOAc, water and brine were added and the organic phase was collected, dried (Mg₂SO₄), filtered and purified by preparative HPLC to give the title compound (8 mg, 10% yield). ¹H NMR (500 MHz, DMSO-d₆) δ ppm 0.98 (t, 3H), 1.16 (t, 3H), 2.11 (s, 3H), 2.35 (m, 2H), 3.61-4.06 (m, 2H), 6.71 (br. s, 2H), 7.09-7.24 (m, 1H), 7.24-7.36 (m, 2H), 7.38-7.48 (m, 1H), 7.47-7.66 (m, 1H,) 7.80 (d, 1H), 7.91 (s, 1H), 8.30 (s, 1 H), 8.61 (m, 2H), 9.11 (br. s, 1H); MS (ES+) m/z 492 [M+H]⁺.

### Example 17

### 5-(3-(3-Amino-1-(1,5-dimethyl-6-oxo-1,6-dihydropyridin-3-yl)-4-fluoro-1H-isoindol-1-yl)phenyl)nicotinonitrile

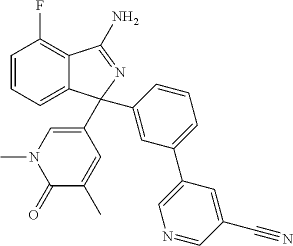

5-(3-Amino-1-(3-bromophenyl)-4-fluoro-1H-isoindol-1-yl)-1,3-dimethylpyridin-2(1H)-one (180 mg, 0.42 mmol, Example 91 method B), 5-cyanopyridin-3-ylboronic acid (81 mg, 0.55 mmol), [1,1-bis(diphenylphosphino)ferrocene]dichloropalladium(II) (31 mg, 0.04 mmol), potassium carbonate (2 M, aq.) (0.633 mL, 1.27 mmol) and DMF (3 mL) were added to a vial and microwaved for 15 min at 150° C. The reaction mixture was diluted with brine, NaHCO₃ (aq. sat.) and EtOAc. The phases were separated, the aqueous phase was extracted with EtOAc (×6), the combined organics were dried (Na₂SO₄), filtered and concentrated. Purification by is preparative chromatography gave the title compound (31 mg, 16% yield). ¹H NMR (500 MHz, DMSO-d₆) δ ppm 1.92 (s, 3H), 3.36 (s, 3H), 6.54 (br. s., 2H), 7.21-7.29 (m, 3H), 7.42-7.56 (m, 3H), 7.63-7.76 (m, 3H), 8.55 (t, 1H), 9.00 (d, 1H), 9.07 (d, 1H); MS (ES+) m/z 450 [M+H]⁺.

### Example 18

### 5-(3-Amino-1-(3-(5-chloropyridin-3-yl)phenyl)-4-fluoro-1H-isoindol-1-yl)-1,3-dimethylpyridin-2(1H)-one

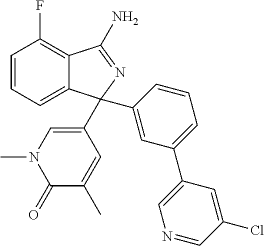

5-(3-Amino-1-(3-bromophenyl)-4-fluoro-1H-isoindol-1-yl)-1,3-dimethylpyridin-2(1H)-one (180 mg, 0.42 mmol, Example 91 method B), 5-chloropyridin-3-ylboronic acid (86 mg, 0.55 mmol), [1,1-bis(diphenylphosphino)ferrocene]dichloropalladium(II) (31 mg, 0.04 mmol), potassium carbonate (2 M, aq.) (0.633 mL, 1.27 mmol) and DMF (3 mL) were added to a vial, and microwaved for 15 min at 150° C. The reaction mixture was diluted with brine, NaHCO₃ (aq. sat.) and EtOAc. The phases were separated and the aq. phase was extracted with EtOAc (x 3). The combined organics were dried (Na₂SO₄), filtered and concentrated. Purification by preparative chromatography gave the title compound (61 mg, 31% yield). ¹H NMR (500 MHz, DMSO-d₆) δ ppm 1.93 (s, 3H), 3.36 (s, 3H), 6.54 (br. s., 2H), 7.21-7.29 (m, 3H), 7.41-7.49 (m, 2H), 7.52 (td, 1H), 7.60-7.66 (m, 2H), 7.71 (d, 1H), 8.12 (t, 1H), 8.61 (d, 1H), 8.74 (d, 1 H); MS (ES+) m/z 459 [M+H]⁺.

### Example 19

### 5-(3-Amino-4-fluoro-1-(3-(5-fluoropyridin-3-yl)phenyl)-1H-isoindol-1-yl)-1,3-dimethylpyridin-2(1H)-one

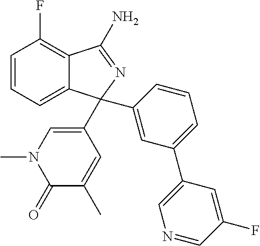

5-(3-Amino-1-(3-bromophenyl)-4-fluoro-1H-isoindol-1-yl)-1,3-dimethylpyridin-2(1H)-one (180 mg, 0.42 mmol, Example 9i method B), 5-fluoropyridin-3-ylboronic acid (77 mg, 0.55 mmol), [1,1-bis(diphenylphosphino)ferrocene]dichloropalladium(II) (31 mg, 0.04 mmol), potassium carbonate (2 M, aq.) (0.633 mL, 1.27 mmol) and DMF (3 mL) were added to a vial, and microwaved for 15 min at 150° C. The reaction mixture was diluted with brine, NaHCO₃ (aq. sat.) and EtOAc. The phases were separated. The aqueous phase was extracted with EtOAc (×3), the combined organics were dried (Na₂SO₄), filtered and concentrated. Purification by preparative chromatography gave the title compound (56 mg, 30% yield). ¹H NMR (500 MHz, DMSO-d₆) δ ppm 1.93 (s, 3H), 3.36 (s, 3H), 6.54 (br. s., 2H), 7.21-7.29 (m, 3H), 7.41-7.48 (m, 2H), 7.52 (td, 1H), 7.60-7.66 (m, 2H), 7.71 (d, 1H), 7.92-7.99 (m, 1H), 8.57 (d, 1H), 8.67 (t, 1H); MS (ES+) m/z 443 [M+H]⁺.

### Example 20

### 5-(3-amino-4-fluoro-1-(3-(5-methoxypyridin-3-yl)phenyl)-1H-isoindol-1-yl)-1,3-dimethylpyridin-2(1H)-one

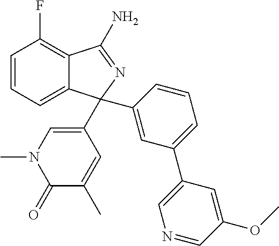

5-(3-Amino-1-(3-bromophenyl)-4-fluoro-1H-isoindol-1-yl)-1,3-dimethylpyridin-2(1H)-one (180 mg, 0.42 mmol, Example 9i method B), 3-methoxy-5-(4,4,5,5-tetramethyl-1,3,2-dioxaborolan-2-yl)pyridine (129 mg, 0.55 mmol), [1,1-bis(diphenylphosphino)ferrocene]dichloropalladium(II) (31 mg, 0.04 mmol), potassium carbonate (2 M, aq.) (0.633 mL, 1.27 mmol) and DMF (3 mL) were added to a vial, and microwaved for 15 min at 150° C. The reaction mixture was diluted with brine and EtOAc, the phases were separated. The aqueous phase was extracted with EtOAc (×2), NaHCO₃ (aq. sat.) was added. The mixture was extracted once more with EtOAc. The combined organics were dried (Na₂SO₄), filtered and concentrated. Purification by preparative chromatography gave the title compound (59 mg, 31% yield). ¹H NMR (500 MHz, DMSO-d₆) δ ppm 1.93 (s, 3H), 3.36 (s, 3H), 3.88 (s, 3H), 6.54 (br. s., 2H), 7.21-7.31 (m, 3H), 7.38-7.44 (m, 2H), 7.48 (dd, 1H), 7.52 (td, 1H), 7.56-7.62 (m, 2H), 7.68 (d, 1H), 8.28 (d, 1H), 8.35 (d, is 1H); MS (ES+) m/z 455 [M+H]⁺.

### Example 21

### (R) and (S) 5-(3-Amino-4-fluoro-1-(4-fluoro-3-(5-(prop-1-ynyl)pyridin-3-yl)phenyl)-1H-isoindol-1-yl)-1,3-dimethylpyridin-2(1H)-one

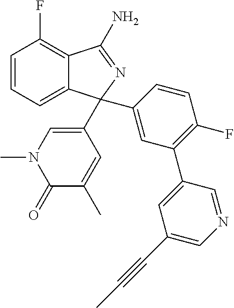

The title compounds were synthesized as described for Example 1 starting from 5-(prop-1-ynyl)pyridin-3-ylboronic acid (0.361 g, 2.24 mmol, Example 28i) and 5-(3-amino-1-(3-bromo-4-fluorophenyl)-4-fluoro-1H-isoindol-1-yl)-1,3-dimethylpyridin-2(1H)-one (0.664 g, 1.49 mmol, Example 21i). Chromatography on a silica gel column eluted with a gradient of 0-3.5% 0.1 M NH₃ in MeOH in DCM gave the racemic mixture of the title compound (0.6 g, 84% yield). ¹H NMR (400 MHz, DMSO-d₆) δ ppm 1.93 (s, 3H), 2.10 (s, 3H), 3.35 (s, 3H), 6.57 (br. s., 2H), 7.22-7.32 (m, 4H), 7.44-7.56 (m, 3H), 7.70 (m, 1H), 7.89 (m, 1H), 8.60 (m, 2H); MS (ES+) m/z 481 [M+H]⁺.

Chromatographic separation of the enantiomers of 5-(3-amino-4-fluoro-1-(4-fluoro-3-(5-(prop-1-ynyl)pyridin-3-yl)phenyl)-1H-isoindol-1-yl)-1,3-dimethylpyridin-2(1H)-one: The sample was dissolved in 2-propanol and injected on a Chiralpak AD-H column (30×300 mm), using 2-propanol (containing 0.1% DEA)/n-heptane (30:70) as eluent at a flow rate of 120 mL/min. Detection was monitored at 220 nm and the two isomers were collected and concentrated in vacuo.

Isomer 1, 130 mg was collected at retention time 8.5 min, 99% enantiomerically pure with unknown absolute configuration: ¹H NMR (500 MHz, DMSO-d₆) δ ppm 1.93 (s, 3H), 2.10 (s, 3 is H), 3.35 (s, 3H), 6.57 (br. s., 2H), 7.22-7.31 (m, 4H), 7.44-7.50 (m, 2H), 7.53 (m, 1H), 7.70 (d, 1H), 7.89 (s, 1H), 8.57-8.62 (m, 2H); MS (ES+) m/z 481 [M+H]⁺; and

Isomer 2, 145 mg was collected at retention time 16 min, 99% enantiomerically pure with unknown absolute configuration: ¹H NMR (500 MHz, DMSO-d₆) δ ppm 1.93 (s, 3H), 2.10 (s, 3 H), 3.35 (s, 3H), 6.57 (br. s., 2H), 7.23-7.31 (m, 4H), 7.44-7.50 (m, 2H), 7.53 (m, 1H), 7.70 (d, 1H), 7.87-7.91 (m, 1H), 8.60 (m, 2H); MS (ES+) m/z 481 [M+H]⁺.

### Example 22

### 5-(3-Amino-4-fluoro-1-(3-(5-fluoropyridin-3-yl)phenyl)-1H-isoindol-1-yl)-3-(difluoromethyl)-1-ethylpyridin-2(1H)-one

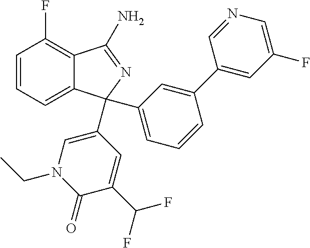

5-(3-Amino-1-(3-bromophenyl)-4-fluoro-1H-isoindol-1-yl)-3-(difluoromethyl)-1-ethylpyridin-2(1H)-one (70 mg, 0.15 mmol, Example 26i), 5-fluoropyridine-3-boronic acid (60 mg, 0.43 mmol), [1,1′-bis(diphenylphosphino)ferrocene]palladium(II) chloride (7 mg, 8.57 μmol), cesium carbonate (144 mg, 0.44 mmol) and DME:EtOH:water (6:3:1) (2.00 mL) were put in a microwave vial and heated at 150° C. in a microwave reactor for 20 min. The reaction mixture was purified by preparative HPLC to give the title compound (44 mg, 61% yield): ¹H NMR (400 MHz, DMSO-d₆) δ ppm 1.17 (t, 3H), 3.92 (m, 2H), 6.50-6.99 (m, 3H), 7.27 (dd, 1H), 7.41-7.50 (m, 2H), 7.55 (td, 1H), 7.59-7.69 (m, 4H), 7.74 (d, 1H), 7.97 (m, 1H), 8.58 (d, 1H), 8.69 (t, 1H); MS (ES+) m/z 493 (M+H)⁺, MS (ES−) m/z 491 [M−Hr]⁻.

### Example 23

### 5-(3-Amino-4-fluoro-1-(3-(5-(prop-1-ynyl)pyridin-3-yl)phenyl)-1H-isoindol-1-yl)-3-(difluoromethyl)-1-ethylpyridin-2(1H)-one

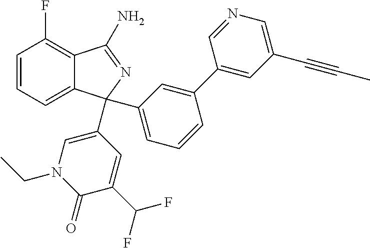

The title compound was synthesized as described for Example 22 in 37% yield starting from 5-(3-amino-1-(3-bromophenyl)-4-fluoro-1H-isoindol-1-yl)-3-(difluoromethyl)-1-ethylpyridin-2(1H)-one (68 mg, 0.14 mmol, Example 26i) and 5-(prop-1-ynyl)pyridin-3-ylboronic acid (33 mg, 0.21 mmol, Example 28i), with the exception that the product was purified by preparative HPLC followed by flash chromatography on silica gel using a gradient from DCM to 70% (3.5 N NH₃ in 10% MeOH in DCM): ¹H NMR (500 MHz, DMSO-d₆) δ ppm 1.18 (t, 3H), 2.10 (s, 3H), 3.92 (m, 2H), 6.41-6.96 (m, 3H), 7.27 (t, 1H), 7.38-7.48 (m, 2H), 7.55 (td, 1H), 7.59-7.67 (m, 4H), 7.74 (d, 1H), 7.97 (s, 1H), 8.56 (d, 1H), 8.72 (d, 1H); MS (ES+) m/z 513 [M+H]⁺.

### Example 24

### (R)-5-(3-Amino-4-fluoro-1-(3-(6-(prop-1-ynyl)pyridin-2-yl)phenyl)-1H-isoindol-1-yl)-1-ethyl-3-methylpyridin-2(1H)-one

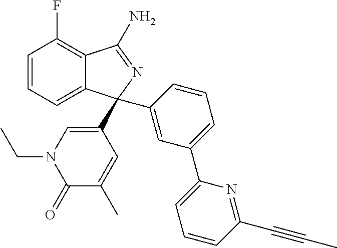

(R)-5-(3-Amino-1-(3-bromophenyl)-4-fluoro-1H-isoindol-1-yl)-1-ethyl-3-methylpyridin-2(1H)-one (110 mg, 0.25 mmol, Example 2i), potassium acetate (49 mg, 0.50 mmol), 1,1′-bis(diphenyl-phosphino)ferrocenedichloro palladium(II) dichloromethane complex (18.3 mg, 0.02 mmol) and bis(pinacolato)diboron (70 mg, 0.27 mmol) in DME (3 mL) were microwaved for 20 min at 130° C. 2-Bromo-6-(prop-1-ynyl)pyridine (54 mg, 0.27 mmol, Example 29i) and water (1 mL) were added. The resulting mixture was microwaved twice for 40 min at 130° C. More 2-bromo-6-(prop-1-ynyl)pyridine (54 mg, 0.27 mmol) and 1,1′-bis(diphenylphosphino)ferrocenedichloro palladium(II) dichloromethane complex (18.3 mg, 0.02 mmol) were added and the mixture was microwaved again for 40 min at 150° C. The mixture was diluted with EtOAc and brine. The aqueous phase was extracted twice with EtOAc, the combined organics were dried (Na₂SO₄), filtered and concentrated. Purification by preparative chromatography gave the title compound (3.3 mg, 3% yield): ¹H NMR (500 MHz, DMSO-d₆) δ ppm 1.15 (t, 3H), 1.93 (s, 3H), 2.09 (s, 3 H), 3.84 (q, 2H), 6.59 (br. s., 2H), 7.20-7.30 (m, 3H), 7.36-7.44 (m, 3H), 7.52 (td, 2H), 7.63 (d, 1H), 7.76 (d, 1H), 7.80-7.90 (m, 2H), 8.01 (s, 1H); MS (ES+) m/z 477 [M+H]⁺.

### Example 25

### (R)-5-(3-Amino-4-fluoro-1-(3-(4-(prop-1-ynyl)pyridin-2-yl)phenyl)-1H-isoindol-1-yl)-1-ethyl-3-methylpyridin-2(1H)-one

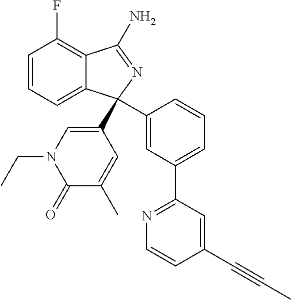

(R)-5-(3-Amino-1-(3-bromophenyl)-4-fluoro-1H-isoindol-1-yl)-1-ethyl-3-methylpyridin-2(1H)-one (50 mg, 0.11 mmol, Example 2i), potassium acetate (22 mg, 0.23 mmol), bis(pinacolato)-diboron (32 mg, 0.12 mmol) and PdCl₂(dppf)-CH₂Cl₂ adduct (4.6 mg, 5.7 μmol) in dioxane (1.0 mL) were heated in a microwave reactor at 110° C. for 20 min. After cooling, potassium carbonate (31 mg, 0.23 mmol), Pd(Ph₃P)₄ (6.6 mg, 5.7 μmol), and water (0.3 mL) were added followed by 2-chloro-4-(prop-1-ynyl)pyridine (19 mg, 0.12 mmol, Example 30i) in dioxane (0.5 mL). The reaction mixture was heated in a microwave reactor at 110° C. for 30 min. After cooling, the reaction mixture was filtered and evaporated. Purification by column chromato-graphy using a gradient of EtOAc in heptane (0-100%), then 1% triethyl amine in EtOAc gave the title compound (15 mg, 28% yield): ¹H NMR (500 MHz, DMSO-d₆) δ ppm 1.14 (m, 3H), 1.93 (m, 3H), 2.12 (s, 3H), 3.84 (q, 2H), 6.57 (br. s., 1H), 7.19-7.27 (m, 3H), 7.30 (dd, 1H), 7.38-7.43 (m, 2H), 7.52 (m, 1H), 7.62 (d, 1H), 7.80 (s, 1H), 7.90 (m, 1H), 8.04 (s, 1H), 8.58 (d, 1H). MS (ES+) m/z 477 [M+H]⁺.

### Example 26

### (R) and (S) 5-(3-amino-4-fluoro-1-(5′-(prop-1-ynyl)-2,3′-bipyridin-4-yl)-1H-isoindol-1-yl)-1-ethyl-3-methylpyridin-2(1H)-one

5-(3-Amino-4-fluoro-1-(5′-(prop-1-ynyl)-2,3′-bipyridin-4-yl)-1H-isoindol-1-yl)-1-ethyl-3-methylpyridin-2(1H)-one (230 mg, 0.48 mmol, Example 2), was dissolved in 2-propanol and injected on a SFC Berger Multigram II system equipped with a Chiralpak AD-H column (20×250 mm, 5 μm), using 2-propanol (containing 0.1% DEA)/CO₂ (25:75) as eluent at a flow rate of 50 mL/min to give:

Isomer 1, with unknown absolute configuration (77 mg, 33% yield) with retention time 5.5 min: is ¹H NMR (500 MHz, DMSO-d₆) δ ppm 1.15 (t, 3H), 1.93 (s, 3H), 2.11 (s, 3H), 3.85 (m, 2H), 6.69 (br. s., 2H), 7.23 (d, 1H), 7.26-7.32 (m, 2H), 7.42 (dd, 1H), 7.56 (td, 1H), 7.81 (d, 1H), 7.91 (s, 1H), 8.30 (t, 1H), 8.59-8.65 (m, 2H), 9.08 (d, 1H). MS (ES+) m/z 478 [M+H]⁺; 99% enantiomerical purity; and

Isomer 2, with unknown absolute configuration: (80 mg, 35% yield) with retention time 7.7 min: ¹H NMR (500 MHz, DMSO-d₆) δ ppm 1.15 (t, 3H), 1.93 (s, 3H), 2.11 (s, 3H), 3.86 (m, 2H), 6.68 (br. s., 2H), 7.23 (d, 1H), 7.26-7.33 (m, 2H), 7.42 (dd, 1H), 7.56 (td, 1H), 7.81 (d, 1H), 7.91 (s, 1H), 8.30 (t, 1H), 8.58-8.66 (m, 2H), 9.08 (d, 1H); MS (ES+) m/z 478 [M+H]⁺; 99% enantiomerical purity.

### Biological Assays

The level of activity of the compounds was tested using the following methods:

### TR-FRET Assay

The β-secretase enzyme used in the TR-FRET is prepared as follows:

The cDNA for the soluble part of the human β-Secretase (AA 1-AA 460) was cloned using the ASP2-Fc10-1-IRES-GFP-neoK mammalian expression vector. The gene was fused to the Fc domain of IgG1 (affinity tag) and stably cloned into HEK 293 cells. Purified sBACE-Fc was stored in −80° C. in Tris buffer, pH 9.2 and had a purity of 40%.

The enzyme (truncated form) was diluted to 6 μg/mL (stock 1.3 mg/mL) and the substrate (Europium)CEVNLDAEFK(Qsy7) to 200 nM (stock 120 μM) in reaction buffer (NaAcetate, is chaps, triton x-100, EDTA pH4.5). The robotic systems Biomek FX and Velocity 11 were used for all liquid handling and the enzyme and substrate solutions were kept on ice until they were placed in the robotic system. Enzyme (9 μA) was added to the plate then 1 μA of compound in dimethylsulphoxide was added, mixed and pre-incubated for 10 minutes. Substrate (10 μA) was then added, mixed and the reaction proceeded for 15 minutes at r.t. The reaction was stopped with the addition of Stop solution (7 μl, NaAcetate, pH 9). The fluorescence of the product was measured on a Victor II plate reader with an excitation wavelength of 340 nm and an emission wavelength of 615 nm. The assay was performed in a Costar 384 well round bottom, low volume, non-binding surface plate (Corning #3676). The final concentration of the enzyme was 2.7 μg/ml; the final concentration of substrate was 100 nM (Km of ˜250 nM). The dimethylsulphoxide control, instead of test compound, defined the 100% activity level and 0% activity was defined by wells lacking enzyme (replaced with reaction buffer). A control inhibitor was also used in dose response assays and had an IC₅₀ of ˜150 nM.

### Diluted TR-FRET Assay

Compounds with a high affinity were further tested in a diluted TR-FRET assay, conditions as described above for the TR-FRET assay, but with 50 times less enzyme and a 6.5 h long reaction time at r.t. in the dark.

sAPPβ Release Assay

SH-SY5Y cells were cultured in DMEM/F-12 with Glutamax, 10% FCS and 1% non-essential amino acids and cryopreserved and stored at −140° C. at a concentration of 7.5−9.5×10⁶ cells per vial. Thaw cells and seed at a conc. of around 10000 cells/well in DMEM/F-12 with Glutamax, 10% FCS and 1% non-essential amino acids to a 384-well tissue culture treated plate, 100 μL cell susp/well. The cell plates were then incubated for 7-24 h at 37° C., 5% CO₂. The cell medium was removed, followed by addition of 30 μL compound diluted in DMEM/F-12 with Glutamax, 10% FCS, 1% non-essential amino acids and 1% PeSt to a final conc. of 1% DMSO. The compounds were incubated with the cells for 17 h (overnight) at 37° C., 5% CO₂. Meso Scale Discovery (MSD) plates were used for the detection of sAPPβ release. MSD sAPPβ plates were blocked in 1% BSA in Tris wash buffer (40 μL/well) for 1 h on shake at r.t. and washed 1 time in Tris wash buffer (40 μL/well). 20 μL of medium was transferred to the pre-blocked and washed is MSD sAPPβ microplates, and the cell plates were further used in an ATP assay to measure cytotoxicity. The MSD plates were incubated with shaking at r.t. for 2 h and the media discarded. 10 μL detection antibody was added (1 nM) per well followed by incubation with shaking at r.t. for 2 h and then discarded. 40 μL Read Buffer was added per well and the plates were read in a SECTOR Imager.

ATP Assay

As indicated in the sAPPβ release assay, after transferring 20 μL medium from the cell plates for sAPPβ detection, the plates were used to analyse cytotoxicity using the ViaLight™ Plus cell proliferation/cytotoxicity kit from Cambrex BioScience that measures total cellular ATP. The assay was performed according to the manufacture's protocol. Briefly, 10 μL cell lysis reagent was added per well. The plates were incubated at r.t. for 10 min. Two min after addition of 25 μL reconstituted ViaLight™ Plus ATP reagent, the luminescence was measured in a Wallac Victor2 1420 multilabel counter. Tox threshold is a signal below 75% of the control.

### Results

Typical IC₅₀ values for the compounds of the present invention are in the range of about 0.1 to about 10,000 nM. Biological data on exemplified final compounds is given below in Table I.

Table I

| | IC₅₀ in TR-FRET | IC₅₀ in sAPPβ release |
|---|---|---|
| Example No. | assay (nM) | assay (nM) |
| 1 | 6^{a} | 2 |
| 2 | 57 | 2 |
| 3 | 2^{a} | 0.2 |
| 4 | 51 | 55 |
| 5 | 58 | 10 |
| 6 | 16^{a} | 9 |
| 7 | 38 | 4 |
| 8 | 8^{a} | 2 |
| 9 | 13^{a} | 4 |
| 10 | 89 | 20 |
| 11 | 54 | 46 |
| 12 | 62 | 8 |
| 13 | 81 | 24 |
| 14 | 58 | 11 |
| 15 | 104 | 14 |
| 16 | 59 | 1 |
| 17 | 66 | 17 |
| 18 | 26 | 8 |
| 19 | 61 | 3 |
| 20 | 31 | 6 |
| 21a | 4250 | 1150 |
| 21b | 18 | 6 |
| 22 | 70 | 9 |
| 23 | 6^{a} | 4 |
| 24 | 140 | 22 |
| 25 | 5^{a} | 2 |
| 26a | 8350 | — |
| 26^{b} | 4^{a} | 0.7 |

^{a}IC₅₀ from the diluted FRET assay.

X-ray Crystal Structure Determination of Example Compounds Co-Crystallized with the Bace1 Protein

### Protein Expression, Purification and Crystallization

Human BACE, CID1328 14-453, was cloned, expressed, refolded, activated and purified according to previously published protocols (Patel, S., Vuillard, L., Cleasby, A., Murray, C. W., Yon, J. J. Mol. Biol. 2004, 343, 407). The protein buffer was exchanged to 20 mM Tris pH 8.5, 150 mM NaCl and concentrated to 3.5 mg/mL. Concentrated protein was mixed 1:1 with a stock of 11% PEG6k, 100 mM Na acetate pH 5.0 at RT and crystallized using vapor diffusion techniques in combination with seeding. The crystals were soaked in a buffer containing 10 mM of the compound in Example 3 or Example 12, 10% DMSO, 18% PEG6k, 90 mM Na acetate pH 4.85, 18 mM Tris pH 8.5 and 135 mM NaCl for 24 hours and flash frozen in liquid nitrogen is using 20% glycerol as a cryoprotectant.

### Data Collection and Refinement

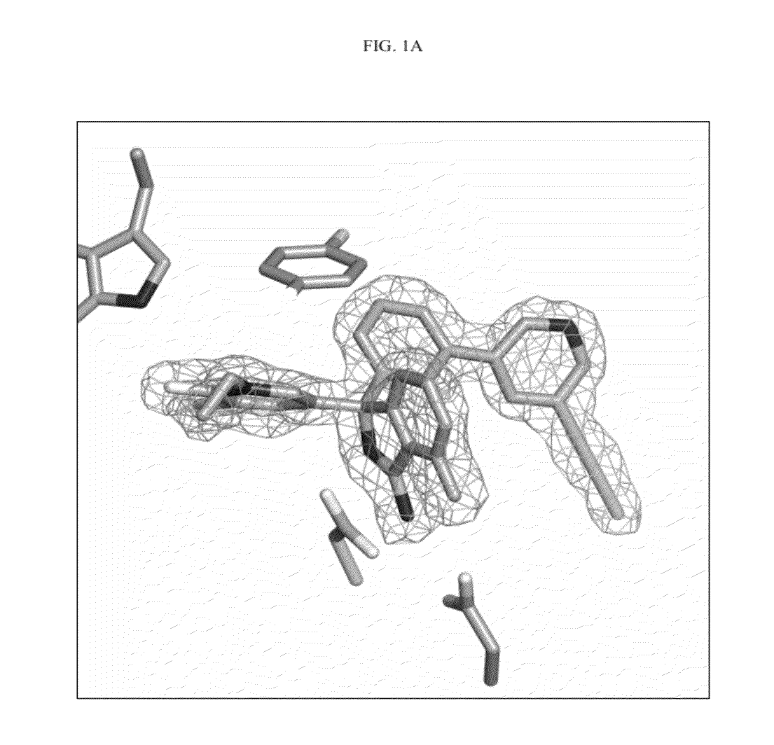

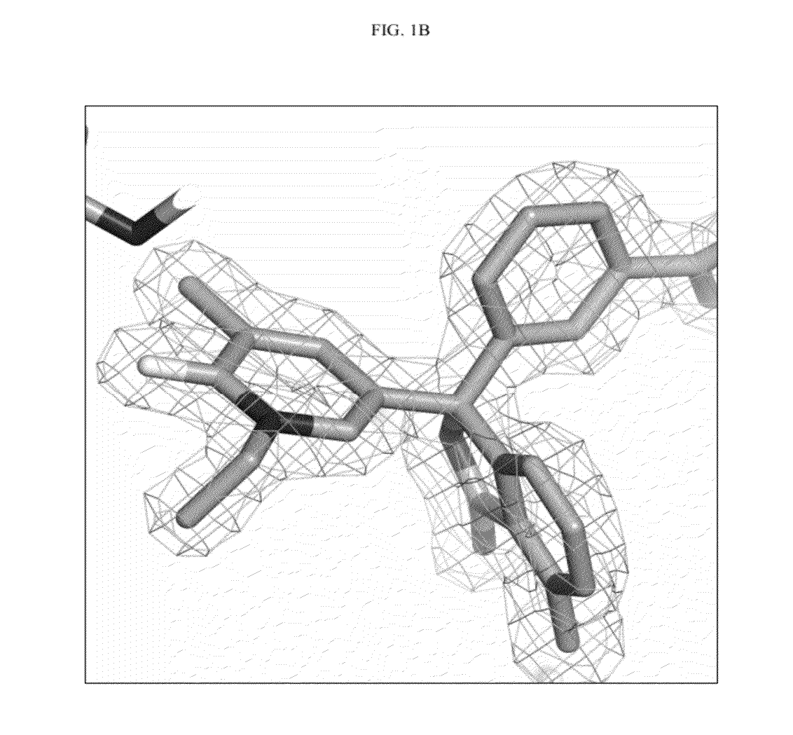

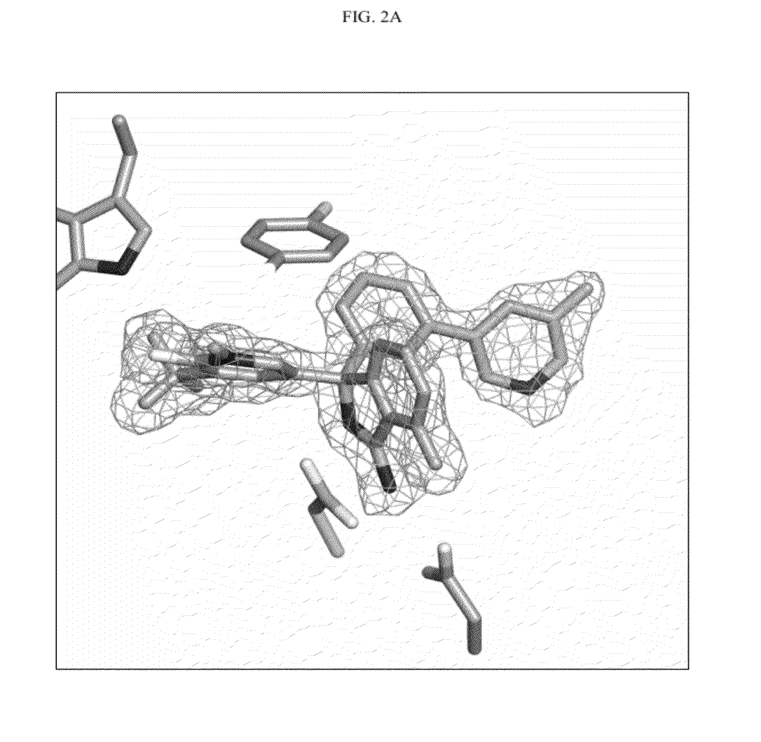

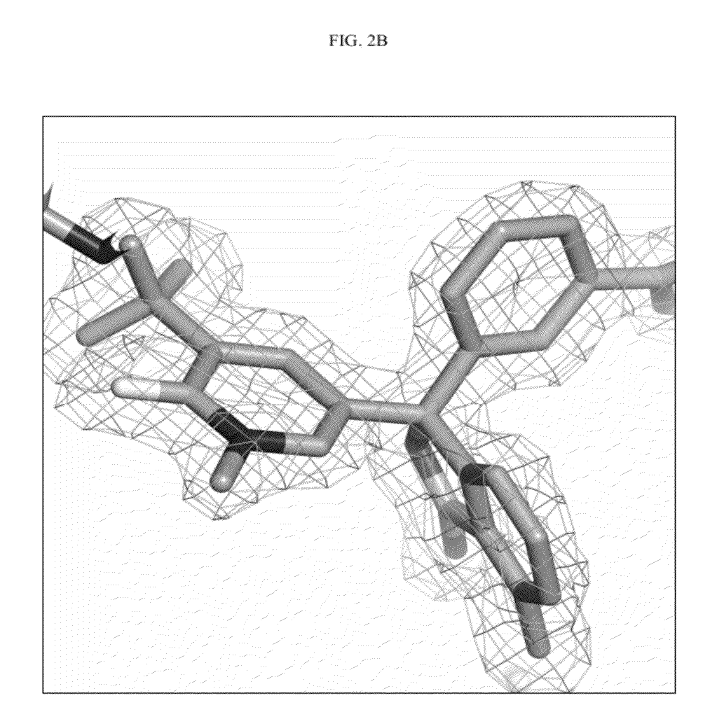

X-ray diffraction data of an Example 3 soaked crystal was collected on a Rigaku FR-E+SuperBright rotating anode and an A200 CCD detector to a resolution of 1.83 Å. Data of compound Example 12 was collected on a Rigaku FR-E rotating anode and a HTC imaging plate to a resolution of 1.85 Å. All data were indexed and integrated with MOSFLM (Leslie, A.G.W. Joint CCP4+ESF-EAMCB Newsletter on Protein Crystallography 1992, 26, 27) and scaled with SCALA (Collaborative Computational Project, Number 4 Acta Crystallogr., Sect. D 1994, 50, 760) in space group P212121, with cell dimensions of about [48,76,105], giving a Matthews coefficient of 2.2 Å³/Da with one monomer per asymmetric unit. The structures of Example 3 and Example 12 were determined by rigid body refinement of a previously determined BACE-1 structure based on the published 1FKN structure (Hong, L., Koelsch, G., Lin, X., Wu, S., Terzyan, S., Ghosh, A. K., Zhang, X. C., Tang, J. Science 2000, 290, 5489, 150-153) using Refmac5 (Murshudov, G. N., Vagin, A. A., Dodson, E. J. Acta Crystallogr., Sect. D 1997, 53, 240). The initial models were further refined by alternative cycles of model rebuilding in Coot (Emsley, P., Cowtan, K. Acta Crystallogr., Sect. D 2004, 60, 2126) and refinement in Refmac5 and AutoBuster (Bricogne, G., Blanc, E., Brandl, M., Flensburg, C., Keller, P., Paciorek, W., Roversi, P., Sharff, A., Smart, O., Vonrhein, C., Womack, T. Global Phasing Ltd, Cambridge, UK 2010). Strong Fo-Fc density in the vicinity of the BACE active site indicated the location of the bound compound. Restraints for the compounds of Example 3 and Example 12 were generated by Writedict (Wlodek S., Skillman A. G., Nicholls A., Acta Crystallogr., Sect. D 2006, 62, 741-749) and used by Flynn (Wlodek S., Skillman A. G., Nicholls A., Acta Crystallogr., Sect. D 2006, 62, 741-749) to determine the absolute stereochemistry of the compound of interest is based on the refined omit maps. Final refinement of the BACE-inhibitor complexes was performed in Refmac5. Resulting 2Fo-Fc maps of Example 3 and Example 12 can be seen in FIGS. 1-2. Full data collection and refinement statistics can be found in Table II.

TABLE II
Data collection and refinement statistics

| | Example 3 | | Example 12 | |
|---|---|---|---|---|
| Data collection | | | | |
| Space group | P2₁2₁2₁ | | P2₁2₁2₁ | |
| Cell dimensions (Å) | 47.96 75.91 104.65 | | 47.95 75.64 104.60 | |
| Resolution (Å) | 1.83-75.91 | | 1.85-32.03 | |
| R_{merge} | 0.051 | (0.452) | 0.060 | (0.446) |
| <I/σI> | 15.0 | (2.1) | 14.8 | (2.5) |
| Completeness (%) | 99.6 | (99.8) | 99.4 | (99.6) |
| Redundancy | 3.0 | (2.9) | 3.4 | (3.4) |

| | | |
|---|---|---|
| Refinement | | |
| Resolution (Å) | 1.83-61.6 | 1.85-32.0 |
| Measured reflections | 101856 | 112197 |
| Unique reflections | 34330 | 33001 |
| R_{work}/R_{free} | 0.186/0.247 | 0.188/0.251 |
| No. atoms | | |
| Protein | 2981 | 2982 |
| Water | 302 | 332 |
| Ligand | 36 | 36 |
| Average B-factors | | |
| Protein (Å²) | 22.8 | 23.5 |
| Water (Å²) | 30.5 | 34.9 |
| Ligand (Å²) | 14.0 | 15.6 |
| Ramachandran outliers (%) | 0.0 | 0.0 |
| R.m.s deviations | | |
| Bond lengths (Å) | 0.011 | 0.012 |
| Bond angles (°) | 1.39 | 1.51 |

¹Values in parentheses refer to highest-resolution shell.

1. A compound according to formula (I): 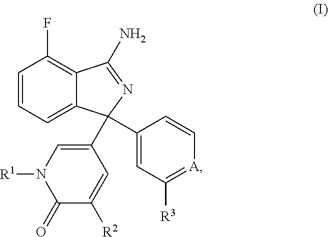
wherein:
A is N or CR⁴;
R¹ is C₁₋₆alkyl, C₃₋₆cycloalkyl or C₁₋₆haloalkyl;
R² is C₁₋₆alkyl, C₃₋₆cycloalkyl or C₁₋₆haloalkyl;
R³ is heteroaryl optionally substituted with one or more R⁵.
R⁴ is hydrogen or halogen;
each R⁵ is independently halogen, cyano, C₁₋₆alkyl, C₁₋₆haloalkyl, C₃₋₆cycloalkyl, C₂₋₆alkenyl, C₂₋₆alkynyl, OC₁₋₆alkyl or OC₁₋₆haloalkyl, wherein:
said C₃₋₆cycloalkyl, C₁₋₆alkyl, C₂₋₆alkenyl, or C₂₋₆alkynyl is optionally substituted with one to three R⁶; and
each R⁶ is independently halogen or OC₁₋₆alkyl;
as a free base or a pharmaceutically acceptable salt thereof.

2. A compound according to claim 1, as a free base or a pharmaceutically acceptable salt thereof, wherein R¹ is C₁₋₆alkyl.

3. A compound according to claim 2, as a free base or a pharmaceutically acceptable salt thereof, wherein R¹ is C₁₋₃alkyl.

4. A compound according to claim 1, as a free base or a pharmaceutically acceptable salt thereof, wherein R² is C₁₋₃alkyl or C₁₋₃haloalkyl.

5. A compound according to claim 4, as a free base or a pharmaceutically acceptable salt thereof, wherein R² is C₁₋₂alkyl or trifluoromethyl.

6. A compound according to claim 1, as a free base or a pharmaceutically acceptable salt thereof, wherein R³ is pyridine or pyrimidine, wherein:
the pyridine or pyrimidine is optionally substituted with one or two R⁵.

7. A compound according to claim 1, as a free base or a pharmaceutically acceptable salt thereof, wherein R⁴ is hydrogen or fluoro.

8. A compound according to claim 1, as a free base or a pharmaceutically acceptable salt thereof, wherein each R⁵ is independently halogen, cyano, C₁₋₃alkyl, C₁₋₃haloalkyl, C₂₋₆alkenyl, C₂₋₆alkynyl or OC₁₋₃alkyl, wherein:
said C₁₋₃alkyl, C₂₋₆alkenyl, or C₂₋₆alkynyl is optionally substituted with one R⁶.

9. A compound according to claim 1, wherein:
A is N or CR⁴;
R¹ is C₁₋₆alkyl or C₁₋₆haloalkyl;
R² is C₁₋₃alkyl or C₁₋₃haloalkyl;
R³ is heteroaryl optionally substituted with one or two R⁵.
R⁴ is hydrogen or fluoro;
each R⁵ is independently halogen, cyano, C₁₋₃alkyl, C₁₋₃haloalkyl, C₂₋₆alkenyl, C₂₋₆alkynyl or OC₁₋₃alkyl, wherein:
said C₁₋₃alkyl, C₂₋₆alkenyl, or C₂₋₆alkynyl is optionally substituted with one to three R⁶; and
each R⁶ is independently halogen or OC₁₋₆alkyl;
as a free base or a pharmaceutically acceptable salt thereof.

10. A compound according to claim 1, wherein:
A is N or CR⁴;
R¹ is C₁₋₃alkyl;
R² is C₁₋₂alkyl or trifluoromethyl;
R³ is pyridine or pyrimidine, wherein:
the pyridine or pyrimidine is optionally substituted with one or two R⁵;
R⁴ is hydrogen or fluoro;
each R⁵ is independently fluoro, chloro, cyano, C₁₋₃alkyl, C₁₋₃haloalkyl, C₂₋₄alkenyl, C₂₋₄alkynyl or OC₁₋₃alkyl, wherein:
said C₁₋₃alkyl, C₂₋₄alkenyl, or C₂₋₄alkynyl is optionally substituted with one R⁶;
each R⁶ is independently halogen or OC₁₋₆alkyl;
as a free base or a pharmaceutically acceptable salt thereof.

11. A compound according to claim 1, as a free base or a pharmaceutically acceptable salt thereof, wherein the stereochemistry of the compound of formula (I) is:

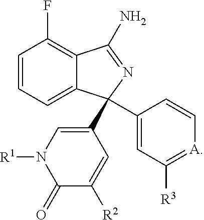

12. A compound according to claim 1, wherein the compound is selected from the group consisting of:
5-(3-Amino-4-fluoro-1-(3-(5-(prop-1-ynyl)pyridin-3-yl)phenyl)-1H-isoindol-1-yl)-1,3-dimethylpyridin-2(1H)-one;
5-(3-Amino-4-fluoro-1-(5′-(prop-1-ynyl)-2,3′-bipyridin-4-yl)-1H-isoindol-1-yl)-1-ethyl-3-methylpyridin-2(1H)-one;
5-(3-Amino-4-fluoro-1-(3-(5-(prop-1-ynyl)pyridin-3-yl)phenyl)-1H-isoindol-1-yl)-1-ethyl-3-methylpyridin-2(1H)-one;
5-(3-Amino-4-fluoro-1-(3-(4-fluoro-5-methylpyridin-3-yl)phenyl)-1H-isoindol-1-yl)-1-ethyl-3-methylpyridin-2(1H)-one;
5-(3-(3-Amino-4-fluoro-1-(1-isopropyl-5-methyl-6-oxo-1,6-dihydropyridin-3-yl)-1H-isoindol-1-yl)phenyl)nicotinonitrile;
5-(3-Amino-4-fluoro-1-(3-(pyrimidin-5-yl)phenyl)-1H-isoindol-1-yl)-1-ethyl-3-methylpyridin-2(1H)-one;
5-(3-Amino-4-fluoro-1-(3-(5-fluoropyridin-3-yl)phenyl)-1H-isoindol-1-yl)-1-isopropyl-3-methylpyridin-2(1H)-one;
5-(3-(3-Amino-1-(1-ethyl-5-methyl-6-oxo-1,6-dihydropyridin-3-yl)-4-fluoro-1H-isoindol-1-yl)phenyl)nicotinonitrile;
5-(3-Amino-4-fluoro-1-(3-(5-fluoropyridin-3-yl)phenyl)-1H-isoindol-1-yl)-1-ethyl-3-methylpyridin-2(1H)-one;
5-(3-(3-Amino-4-fluoro-1-(1-methyl-6-oxo-5-(trifluoromethyl)-1,6-dihydropyridin-3-yl)-1H-isoindol-1-yl)phenyl)nicotinonitrile;
5-(3-Amino-4-fluoro-1-(3-(5-methoxypyridin-3-yl)phenyl)-1H-isoindol-1-yl)-1-methyl-3-(trifluoromethyl)pyridin-2(1H)-one;
5-(3-Amino-4-fluoro-1-(3-(5-fluoropyridin-3-yl)phenyl)-1H-isoindol-1-yl)-1-methyl-3-(trifluoromethyl)pyridin-2(1H)-one;
5-(3-Amino-4-fluoro-1-(3-(pyrimidin-5-yl)phenyl)-1H-isoindol-1-yl)-1-methyl-3-(trifluoromethyl)pyridin-2(1H)-one;
5-(3-Amino-4-fluoro-1-(3-(pyrimidin-5-yl)phenyl)-1H-isoindol-1-yl)-1,3-dimethylpyridin-2(1H)-one;
5-(3-Amino-1-(5′-chloro-2,3′-bipyridin-4-yl)-4-fluoro-1H-isoindol-1-yl)-1,3-dimethylpyridin-2(1H)-one;
5-(3-Amino-4-fluoro-1-(5′-(prop-1-ynyl)-2,3′-bipyridin-4-yl)-1H-isoindol-1-yl)-1,3-diethylpyridin-2(1H)-one;
5-(3-(3-amino-1-(1,5-dimethyl-6-oxo-1,6-dihydropyridin-3-yl)-4-fluoro-1H-isoindol-1-yl)phenyl)nicotinonitrile;
5-(3-Amino-1-(3-(5-chloropyridin-3-yl)phenyl)-4-fluoro-1H-isoindol-1-yl)-1,3-dimethylpyridin-2(1H)-one;
5-(3-Amino-4-fluoro-1-(3-(5-fluoropyridin-3-yl)phenyl)-1H-isoindol-1-yl)-1,3-dimethylpyridin-2(1H)-one;
5-(3-amino-4-fluoro-1-(3-(5-methoxypyridin-3-yl)phenyl)-1H-isoindol-1-yl)-1,3-dimethylpyridin-2(1H)-one;
5-(3-Amino-4-fluoro-1-(4-fluoro-3-(5-(prop-1-ynyl)pyridin-3-yl)phenyl)-1H-isoindol-1-yl)-1,3-dimethylpyridin-2(1H)-one;
5-(3-Amino-4-fluoro-1-(3-(5-fluoropyridin-3-yl)phenyl)-1H-isoindol-1-yl)-3-(difluoromethyl)-1-ethylpyridin-2(1H)-one;
5-(3-Amino-4-fluoro-1-(3-(5-(prop-1-ynyl)pyridin-3-yl)phenyl)-1H-isoindol-1-yl)-3-(difluoromethyl)-1-ethylpyridin-2(1H)-one;
5-(3-Amino-4-fluoro-1-(3-(6-(prop-1-ynyl)pyridin-2-yl)phenyl)-1H-isoindol-1-yl)-1-ethyl-3-methylpyridin-2(1H)-one; and
5-(3-Amino-4-fluoro-1-(3-(4-(prop-1-ynyl)pyridin-2-yl)phenyl)-1H-isoindol-1-yl)-1-ethyl-3-methylpyridin-2(1H)-one;
as a free base or a pharmaceutically acceptable salt thereof.

13. A pharmaceutical composition, wherein the composition comprises:
a therapeutically effective amount of a compound according to claim 1, as a free base or a pharmaceutically acceptable salt thereof; and
at least one pharmaceutically acceptable excipient, carrier or diluent.

14-17. (canceled)

18. A method of treating an Aβ-related pathology in a patient in need thereof, wherein the method comprises administering to said patient a therapeutically effective amount of a compound according to claim 1, as a free base or a pharmaceutically acceptable salt thereof.

19. The method of claim 18, wherein said Aβ-related pathology is Down's syndrome, a β-amyloid angiopathy, cerebral amyloid angiopathy, hereditary cerebral hemorrhage, a disorder associated with cognitive impairment, MCI (“mild cognitive impairment”), Alzheimer's disease, memory loss, attention deficit symptoms associated with Alzheimer's disease, neurodegeneration associated with Alzheimer's disease, dementia of mixed vascular origin, dementia of degenerative origin, pre-senile dementia, senile dementia, dementia associated with Parkinson's disease, progressive supranuclear palsy or cortical basal degeneration.

20. A method of treating Alzheimer's disease in a patient in need thereof, wherein the method comprises administering to said patient a therapeutically effective amount of a compound according to claim 1, as a free base or a pharmaceutically acceptable salt thereof.

21. A method of treating an Aβ-related pathology in a patient in need thereof, wherein the method comprises administering to said patient:
a therapeutically effective amount of a compound according to claim 1, as a free base or a pharmaceutically acceptable salt thereof; and
at least one cognitive enhancing agent, memory enhancing agent, or choline esterase inhibitor.
